(19) United States
(12) Patent Application Publication
(10) Pub. No.: US 2021/0275630 A1
(43) Pub. Date: Sep. 9, 2021

(54) INACTIVATION OF DNA REPAIR AS AN ANTICANCER THERAPY

(71) Applicant: Phoremost Limited, Cambridge (GB)

(72) Inventors: Alberto Bardelli, Torino (IT); Giovanni GERMANO, Torino (IT); Chris TORRANCE, Torino (IT)

(21) Appl. No.: 16/094,072

(22) PCT Filed: Apr. 18, 2017
(86) PCT No.: PCT/GB2017/051062

§ 371 Date: Oct. 16, 2018

(30) Foreign Application Priority Data
Apr. 18, 2016 (GB) 1606721.7
Nov. 18, 2016 (GB) 1619524.0

(51) Int. Cl. A61K 38/17; C07K 16/28; A61K 31/495; G01N 33/50; A61K 38/45; A61K 45/06

(52) U.S. Cl. CPC A61K 38/1709; C07K 16/2818; C07K 16/2827; C12Y 207/07007; G01N 33/5011; A61K 38/45; A61K 45/06; A61K 31/495

(57) ABSTRACT

This invention relates to the modulation of DNA repair and nucleic acid editing mechanisms for use in the treatment of cancer. This invention relates to the inactivation of DNA repair and mechanisms for use in the treatment of cancer. This invention also relates to screening for new anti-cancer agents.

### TECHNICAL FIELD OF THE INVENTION

This invention relates to the modulation of DNA repair and nucleic acid editing mechanisms for use in the treatment of cancer. This invention relates to the inactivation of DNA repair and mechanisms for use in the treatment of cancer. This invention also relates to screening for new anti-cancer agents.

### BACKGROUND OF THE INVENTION

Despite there being a wide range of cancer treatments already available, there is still a need to identify new targets and mechanisms for new anti-cancer agents which provide an increased success rate in treating cancer.

In addition, when metastatic cancers are challenged with anti-cancer targeted agents almost invariably a subset of cells insensitive to the drug emerges. As a result, in most instances, targeted therapies are only transiently effective in patients. Strategies to prevent or overcome resistance are therefore essential to design the next generation of clinical trials. Overcoming the near-certainty of disease recurrence following treatment with targeted agents remains a major problem for cancer treatment.

Accordingly, in addition to new anti-cancer targeted agents for a first line of treatment for newly diagnosed cancer patients, there is also a need to identify new targets and mechanisms to limit the emergence of drug resistance and lead to long-term efficacious therapeutic responses.

### SUMMARY OF THE INVENTION

The present inventors have found that inactivation of a DNA repair gene, exemplified herein by the MMR gene MLH1, in mouse model tumour cell lines resulted in the inability of those cell lines to form tumours when injected into immunocompetent syngeneic mice. Whilst not wishing to be bound by any theory, the inventors have established that tumour-forming ability was restored when host CD-8 T-cells were concomitantly suppressed indicating a role for the host immune system in tumour growth suppression. The present inventors have found that, in cells with an inactivated DNA repair gene, exemplified by a MMR gene, the DNA mutation (and therefore the corresponding neo-antigen) profiles dynamically evolve over time. This would lead to progressive and repeated engagement of the host immune system. This leads to a novel strategy for a first line tumour therapy. In addition, the possible development of resistance to an existing anti-cancer agent is therefore counterbalanced by the dynamic emergence of new antigens that are engaged by new pools of T cells.

While exemplified by a DNA repair gene, the invention may also relate to any genes or protein products whose inactivation or modulation leads to an increase in mutational rates or loads, such as an increase in dynamic mutational loads, or to an increase in neoantigen creation. For example, as the function of several nucleic acid editing enzymes affects the abundance and variety of antigens presented to the immune system, modulation of these enzymes may also favourably increase the propensity of the host immune system to mediate an anti-response to tumours.

Accordingly, in a first aspect, there is provided a method for treating cancer comprising:

a) providing i) a subject having cancerous cells, and ii) a modifier of a gene or its protein product, wherein said gene is one whose modulation leads to a therapeutically favourable increase in antigen-based recognition of tumour tissues by T-cells; and

b) treating said subject with said modifier;

wherein said treating reduces the number of cancerous cells in said subject.

Suitably the gene as defined in part (a) above may be any gene (or its protein product) involved in enzymatic mechanisms such as nucleic acid editing, repair or modification. Suitable editing enzymes include ADAR family enzymes involved in RNA editing, and the APOBEC or AICDA family enzymes that edit DNA.

Accordingly, in another aspect, there is provided a method for treating cancer comprising:

a) providing i) a subject having cancerous cells, and ii) a modifier of a DNA repair or nucleic acid editing gene or its protein product; and

b) treating said subject with said modifier;

wherein said treating reduces the number of cancerous cells in said subject.

In another aspect, there is provided a method for treating cancer comprising:

a) providing i) a subject having cancerous cells, and ii) a modifier of a DNA repair gene or its protein product; and

b) treating said subject with said modifier;

wherein said treating reduces the number of cancerous cells in said subject.

Suitably a reduction in the number of cancerous cells in a subject may be determined by detecting a reduction in tumour mass or size. A reduction in the number of cancerous cells may also be determined by any clinical endpoint which indicates a successful cancer therapy e.g. an absence of tumour relapse or recurrence or an increase in survival rate compared to the average survival rate observed in similar individuals in the absence of said treatment.

In one embodiment, the subject having cancerous cells is a subject which has a tumour which is proficient in DNA repair or nucleic acid editing i.e. it is not a tumour in which a DNA repair or nucleic acid editing deficiency has already been identified. Methods for identifying a DNA repair or nucleic acid editing deficiency in a tumour sample will be familiar to those skilled in the art. In particular embodiments therefore, the subject having cancerous cells is a subject which has a tumour which does not have a MLH-1 deficiency, for example.

In one embodiment, the modifier of a DNA repair or nucleic acid editing gene, or its protein product, is an activator of a DNA repair or nucleic acid editing gene, or its protein product. In this embodiment, the DNA repair gene may be selected from DNA polymerases including those involved in translesion synthesis such as, for example, DNA pol η, ι and κ. The nucleic acid editing gene may be selected from enzymes that edit or alter DNA or RNA in a fashion that leads to the increased presence or expression of mutant gene products. Such a nucleic acid editing gene may be an RNA editing enzyme. Suitable genes here include, for example, ADAR (Adenosine Deaminase, RNA-Specific) enzymes and APOBEC enzymes such as APOBEC1, APOBEC3A, APOBEC3B, AICDA.

In another embodiment, the modulator of a DNA repair or nucleic acid editing gene or its protein product is an inactivator of the gene or its protein product.

Examples of DNA repair genes are given herein. In one embodiment, the DNA repair gene is an MMR gene such as, for example, a MutL homologue. Suitable MutL homologues include, for example, MLH1, MutLα, MutLβ, MutLγ, PMS1, PMS2 or MLH3. In one embodiment, the DNA repair gene is MLH1. In another embodiment, the DNA repair gene may be a proof reading DNA polymerase such as, for example, POLE, POLD or POLQ or a homologous recombination enzyme such as BRCA1 or BRCA2.

Examples of nucleic acid editing genes are given herein.

Suitably a “modifier” for use in accordance with any aspect or embodiment of the invention is a polypeptide, polynucleotide, antibody, peptide or small molecule compound.

In one embodiment of any aspect of the invention, an modifier of a DNA repair or nucleic acid editing gene may be a molecule which provides modulation through altering the gene at the level of modifying expression of the gene by altering its genetic code. Suitable methods for modifying gene expression are known to those skilled in the art and include using genome editing methods. For example, a gene may be knocked-out or modulated using a CRISPR-based genome editing approach. Suitable methods for genome editing are described herein. In one embodiment, those specific genome editing constructs described herein may be used for a method in accordance with the invention. Other methods for knocking out or modifying gene expression from a particular gene include using an interfering RNA approach.

In one embodiment of any aspect of the invention, an inactivator of a DNA repair gene may be a molecule which provides inactivation through inactivating the gene at the level of silencing or knocking out the expression of the gene. Suitable methods for knocking out gene expression are known to those skilled in the art and include using genome editing methods. For example, a gene may be knocked-out using a CRISPR-based genome editing approach. Suitable methods for genome editing are described herein. In one embodiment, those specific genome editing constructs described herein may be used for a method in accordance with the invention. Other methods for knocking out or reducing gene expression from a particular gene include using an interfering RNA approach.

In one embodiment, the cancer for treatment in accordance with the invention is a MMR +ve cancer.

In one embodiment the invention provides a method for treating cancer wherein the modifier of a DNA repair or nucleic acid editing gene, or its protein product, is provided as part of a treatment in combination with another, different or second, cancer treatment. Accordingly, there is provided a method of treatment of cancer in a subject in need thereof comprising administering to said subject a therapeutically effective amount of a) a modifier of a DNA repair gene, or its protein product, and b) a different (i.e. a further or second) cancer treatment. The different/other (i.e. further/second) cancer treatment may be provided separately, simultaneously or sequentially. Suitably, the different cancer treatment is one which inactivates other DNA repair mechanisms. e.g. by inactivating MMR genes. Compounds which inactivate MMR genes will be familiar to those skilled in the art and include, for example, temozolomide (TMZ) or MNU or their derivatives. In the case of temozolomide and other similar compounds, it may be recognised that it does not directly inactivate DNA repair but that acquired resistance to TMZ exposure results in inactivation of DNA repair.

In another embodiment the different cancer treatment may be an immunotherapy i.e. a therapy that uses the immune system to treat cancer. A wide range of immunotherapy approaches will be known to those skilled in the art. In particular, compounds (e.g. peptides, antibodies, small molecules and so forth) that act as immune checkpoints, i.e. affect immune system functioning may be used in combination with a method of treatment in accordance with the invention. For example, an immune checkpoint therapy may block inhibitory checkpoints so as to restore immune system function. Suitable targets for compounds that act on immune checkpoints include, for example, programmed cell death 1 protein (PDCD1, PD-1; also known as CD279) and its ligand, PD-1 ligand 1 (PD-L1, CD274). PD-L1 plays a key regulatory role on T cell activity and cancer-mediated upregulation of PD-L1 on the cell surface has been observed to inhibit T cells which might otherwise attack a cancer cell. Therapeutic antibodies have been developed to bind to either PD-1 or PD-L1 to allow T-cells to attack the tumour by blocking this inhibitory action. Suitable compounds for use in combination with a method of treatment in accordance with the invention therefore include therapeutic antibodies which inhibit PD-1 pathways such as an anti-PD1 antibody (e.g. Nivolumab, Pembrolizumab) and anti-PDL-1 antibodies. Other antibodies include those targeting CTLA-4 e.g. anti-CTLA-4 antibodies. Other immune checkpoint therapies may be developed to similar immune checkpoint targets. In one embodiment the immunotherapy for use in combination with the modifier of DNA repair may be a combination of molecules targeting the immune system e.g. anti-PD1 in combination with anti-CTLA-4 or anti-PDL-1 and so forth.

In one embodiment of the invention, the inactivator of a mismatch repair gene is temozolomide, MNU or their derivatives.

In another aspect, the invention provides a method for screening for anti-cancer compounds comprising

a) providing cells expressing a DNA repair gene,

b) incubating said cells in the presence of a test compound,

c) measuring the rate of DNA mutation in the presence of a test compound;

d) wherein an increased rate of DNA mutation in cells in the presence of a test compound compared to that measured in cells in the absence of a test compound indicates a test compound is an anti-cancer compound.

The invention also provides a method for screening for anti-cancer compounds comprising

a) providing cells expressing a DNA repair or nucleic acid editing gene,

b) incubating said cells in the presence of a test compound,

c) measuring the rate of DNA mutation in the presence of a test compound;

d) wherein an increased rate of DNA mutation in cells in the presence of a test compound compared to that measured in cells in the absence of a test compound indicates a test compound is an anti-cancer compound.

Suitably in the method in accordance with these aspects the cells expressing a DNA repair or nucleic acid editing gene as set out in step a) may further comprise a reporter construct placed out of frame in a construct downstream of a simple nucleotide sequence.

In one embodiment, an increased mutation rate is identified as an increased read-out from the reporter construct in the presence of the test compound compared to the read-out in the absence of the test compound.

In another aspect, there is therefore provided a method for screening for a modifier of a DNA repair or nucleic acid editing gene, or its protein product, comprising:
a) providing a construct wherein comprising a simple nucleotide sequence cloned upstream of a reporter coding sequence such that the reporter coding sequence is out of frame;
b) transfecting cells with the construct of a) in combination with a construct comprising a DNA repair or nucleic acid editing gene
c) incubating said cells in the presence of a test compound;
d) measuring a signal from the reporter construct;
e) wherein an increase in signal from the reporter construct in the presence of the test compound compared to the signal in the absence of the test compound indicates that the test compound is a modifier of said DNA repair or nucleic acid editing gene, or its protein product.

Suitably the “simple nucleotide sequence” may be any genomic repeat sequence which is known to be a site for replicative errors, for example, one that is known to accumulate mismatches during DNA replication. Suitable genomic repeat sequences include those sequences which are identified as a microsatellite region such as, for example, a microsatellite repeat or a sequence which is associated or indicative of a replicative repair deficiency. Suitable such sequences include poly A sequences such as A₍₁₇₎. In another embodiment, a dinucleotide repeat sequence may be used such as CA or GT, in particular CA₍ₙ₎ where n may be any number. In one embodiment, the dinucleotide repeat sequence is CA₍₁₄₎ or CA₍₂₀₎, also referred to as a CA₍ₙ₎ repeat “tract” sequence. Other repeat sequences such as trinucleotide repeats are also envisaged.

Suitably the reporter coding sequence is a nucleic acid sequence which encodes a reporter moiety. Suitable reporter moieties will be familiar to those skilled in the art and include selectable markers. Examples of reporter moieties include beta-galactosidase, NanoLuc® and so forth. Advantageously, the reporter moiety is one which has a large and linear dynamic range such that a small change in the number of in-frame reporter moieties expressed results in a positive signal, thus allowing a sensitive assay. Suitable selectable markers will be familiar to those skilled in the art and include antibiotic resistance genes and drug selection markers such those genes encoding resistance to antibiotics such as puromycin, G418, hygromycin, blasticidin, puromycin, zeocin or neomycin, for example. Advantageously using a selectable marker allows a survival signal to be detected i.e. only those cells which a test compound acts as a modifier of a DNA repair or nucleic acid editing gene, or its protein product will survive when grown in the presence of an antibiotic.

Suitably the cells for use in a screening method in accordance with the invention are a mammalian cell line. Suitable mammalian cell lines include HEK293 cells such as HEK293A, FT or T cells although other cell lines are envisaged.

Suitable DNA repair or nucleic acid editing genes for use in a screening method in accordance with the invention are described herein and include, for example, those genes which encoded DNA repair enzymes involved in post-replicative DNA repair, such as those genes encoding MMR enzymes, including, for example, MLH-1.

In these aspects, a test compound may be a candidate anti-cancer compound because it is effective to either reduce or modify expression of a DNA repair or nucleic acid editing gene or to act as an inhibitor or modifier of the protein product of a DNA repair gene so as to inhibit or alter DNA repair activity, or otherwise dynamically generate an increased cellular mutation burden. Examples of suitable screening methods are described herein along with examples of suitable methods for measuring the rate of DNA mutation in the Examples section. DNA mutation may be measured as a number of mutations/megabase (Mb) of DNA. For example, functional inactivation of a DNA mismatch repair or nucleic acid editing enzyme may be determined by sequencing repetitive DNA elements or cDNAs. Exome sequencing of cells treated with a test compound compared with untreated cells may be used to measure mutational loads. For example, exome sequencing from cells collected longitudinally at distinct time-points can be performed. Importantly, an increased rate of DNA mutation or cDNA epimutation not only leads to an increase in the number of mutations and antigens but also to the acquisition of new mutations over time as a result of DNA repair inactivation or modification or nucleic acid editing modification. This leads to a dynamic hypermutation state. The mutation (and therefore the corresponding neo-antigen) profiles therefore preferably dynamically evolve over time such that the genomic landscape rapidly and dynamically evolves with the continuous emergence of neo-antigens.

In one embodiment, a high mutational load may be observed in treated cells, thus indicating that a test compound is a candidate anti-cancer agent. Suitably, a high mutational load may be expressed as a mutation rate wherein an increased rate of DNA mutation is in the region of 10-100 mutations/megabase of DNA. In another embodiment, an increased mutational burden may be in the region of over 100 mutations/megabase of DNA.

Methods for determining mutation rate are described, for example with reference to FIG. 4A. RNAseq analysis may also be used to identify the proportion of mutated genes that are transcribed and therefore can act as neo-antigens. Microsatellite instability assays may also be used.

In one embodiment of a method for screening in accordance with the invention, cells expressing a DNA repair gene can be a human tumour cell line e.g. colorectal, breast cancer cells.

Suitably, the DNA repair gene is MLH1. In this embodiment, cells that have lost MLH1 expression or MLH1 activity, for example, through inhibition of gene expression or protein activity as a result of treatment with a candidate anti-cancer compound, are insensitive to inhibitors as demonstrated in FIG. 5A. Thus MMR deficient cells are either not affected or are more resistant to a number of anticancer agents such as those listed in Table 2, for example.

In one embodiment, the rate of DNA mutation is an increased rate of dynamic mutational load. Suitably, such an increased rate translates into the expression of neo antigens.

In another aspect, there is provided a method of identifying a patient having a tumour suitable for treatment by immunotherapy comprising:
a) taking a sample of said tumour,
b) analysing said sample to determine the sequence of a DNA repair gene;
c) comparing the sequence of said DNA repair gene in a tumour sample with the sequence in a non-tumour sample;
wherein a defect in the sequence of said DNA repair gene in the tumour sample compared to the sequence of said gene in a non-tumour sample is indicative that said patient has a tumour suitable for treatment by immunotherapy.

In one embodiment, a mutation in a DNA repair gene is detected. Such a “mutation” may be a whole or partial deletion of the DNA repair gene or a point mutation to render it inactive.

In another aspect, a method for identifying a patient having a tumour suitable for treatment by immunotherapy comprises detecting dysfunctional DNA repair through measuring high rates of mutation. In particular, a method is provided which allows a determination to be made from patients' samples as to whether there is a dynamic change of the mutational status.

In a further aspect, the invention provides a method of treating cancer in an individual comprising diagnosing a cancer subtype in the individual based on a high measurement of mutation rate; and treating the individual with an immunotherapeutic composition.

In another aspect there is provided an inactivator of a DNA repair or nucleic acid editing gene wherein said inactivator comprises a construct which interferes with expression of said DNA repair or nucleic acid editing gene. Suitable inactivators comprise CRISPR constructs, such as the CRISPR construct which is an inactivator of MLH-1, as described herein. Further suitable inactivators include vector encoded siRNAs or anti-sense oligonucleotides.

### DETAILED DESCRIPTION OF THE INVENTION

Cancer genes are commonly classified in two major groups: oncogenes and tumour suppressor genes. The majority of oncogenes control key nodes of signalling pathways and are altered by point mutations that constitutively activate their protein counterparts leading to increased cell proliferation.¹ Tumour suppressor genes typically harbour molecular alterations that inactivate their function such as deletions or loss of function mutations.² Many tumour suppressor genes are involved in amending DNA replication errors that occur during cell division.⁴ Alterations in DNA repair genes do not directly promote cell proliferation but are thought to fuel tumorigenesis by increasing mutation rates thus accelerating cancer evolution.⁵ Germline mutations in genes controlling DNA mismatch repair (MMR) are responsible for cancer syndromes such as Hereditary Non Polyposis Colon Cancer (HNPCC). Individuals affected by HNPCC develop tumours at an early age and have an increased lifespan risk of colorectal, endometrial, urinary tract, ovarian and pancreatic tumours.⁶ MMR genes also promote tumour progression when somatically mutated.^{3,6,7} Approximately 20% of sporadic colorectal cancers, 29% of ovarian and 28% of endometrial cancers carry somatic alterations in MMR genes.^{8,9}

In human cells, post replicative DNA mismatch repair is performed by protein complexes, which involve MLH1, MSH2, MSH6 and PMS2.¹⁰ When the MMR machinery is defective, cells accumulate mutations at an increased rate and display characteristic microsatellite instability (MSI). MMR deficient colorectal tumours have peculiar clinical features, which include early onset and rapid progression but favourable prognosis.¹¹ The molecular basis of these apparently contradictory clinical features was previously poorly understood.

In cancer patients, response to targeted chemotherapy agents is often dramatic but short lived and most of the patients relapse in a matter of weeks or months, this may be characterised by partial resistance to the chemotherapy agent. In contrast to chemotherapy with a targeted agent, a most remarkable feature of immunotherapy is the length of the response, which often lasts for years. This therefore reduces the likelihood of disease recurrence and morbidity and motivation for extensive surgery at the point of primary disease diagnosis. A strategy for anticancer therapy that recruits the patient's immune system to attack tumorous cells is therefore an improvement on targeted chemotherapy agents. It is still possible however that tumour cells may develop resistance to immunomodulatory agents. This may be circumvented by a strategy that promotes the continual emergence of new tumour antigens that are engaged by new pools of T cells, continually re-engaging the patient's immune response to attack the tumorous cells.

Therefore, inactivation or modulation of DNA repair or nucleic acid editing mechanisms leading to a dynamic hyper-mutation status that is central to immune surveillance, or inducing a mutator phenotype sensitive to immune surveillance, is a novel strategy for anticancer therapy.

### Modifier

The term modifier refers to a test compound which changes the activity of a DNA repair or nucleic acid editing gene, or its protein product, in the presence of that compound compared to the activity in the absence of that compound. Suitably a “modifier” can be an activator or an inactivator of the DNA repair or nucleic acid editing gene or its protein product. For example activator may be one which enhances the activity a DNA repair or nucleic acid editing gene or its protein product whereas an inactivator may be one which reduces DNA repair or nucleic acid editing activity either through inhibiting the enzymatic activity of the protein encoded by the DNA repair or nucleic acid editing gene or through stabilising covalent enzyme-DNA complexes such that repair cannot take place.

A modifier, as defined above, is a compound which works to modify a component either by acting at the gene, RNA or protein level. In particular, references to a tumour suppressor “gene” such as a DNA repair or nucleic acid editing “gene” as described herein are to the gene per se as well as the protein encoded by the gene (i.e. its protein product).

Methods for determining whether a compound is a modifier of a DNA repair or nucleic acid editing gene/protein include methods for detecting binding to a particular DNA repair or nucleic acid editing gene of interest, functional assays for a particular DNA repair or nucleic acid editing gene/protein which will be familiar to those skilled in the art and methods for detecting a defect in a DNA repair or nucleic acid editing gene/protein through measuring an increase in mutations. Suitable methods for detecting an increase in mutation are described herein and include methods for measuring microsatellite shifts over time.

In other aspects or embodiments of the invention, there is provided a modifier of a DNA repair or nucleic acid editing gene for use in therapy. In other aspects or embodiments, the invention provides a modifier for use in the treatment of cancer. In other aspects or embodiments, the invention provides a use of a modifier of a DNA repair or nucleic acid editing gene in the manufacture of a medicament for use in the treatment of cancer. In some embodiments, the modifier may be used in a combination therapy.

### DNA Repair Mechanisms

In one embodiment of any aspect of the invention, the present invention provides for modifying tumour suppressor genes such as those involved in DNA repair. Suitably, the invention may relate to any genes whose inactivation leads to an increase in mutational rates or loads, such as an increase in dynamic mutational loads.

In cells, DNA is susceptible to many chemical alterations that can lead to mutations and there is a network of genes and their protein products involved in DNA repair mechanisms to correct damaged or inappropriate bases so that mutations do not accumulate. “DNA repair genes” are those genes which encode proteins involved in DNA repair mechanisms. As used herein, the term DNA repair genes refers to the genes and also to the proteins they encode.

DNA repair mechanisms include 1) direct chemical reversal of the damage and 2) Excision Repair. In excision repair, damaged base or bases are removed, then replaced/corrected in a localized area of DNA synthesis. Excision repair includes Base Excision Repair (BER), Nucleotide Excision Repair (NER) and Mismatch Repair (MMR), each of which uses specific sets of enzymes.

A large number of genes are reported to be involved in DNA repair mechanisms. These may be grouped broadly according to function e.g. Non-homologous end joining (NHEJ) genes, including XRCC4, LIG4, DNA-PK; Microhomology mediated end joining (MMEJ) genes, including MREII, XRCC1, LIG3; Homologous Recombination (HR) genes, including BRCA1, BRCA2, RAD51, LIG1; Mismatch repair (MMR) genes, including MLH1, MSH2, PMS2; Base Excision Repair (BER) genes, including Uracil DNA-glycosylase, AP-Endonuclease; Nucleotide Excision Repair (NER) genes, including XPC, XPD, XPA; DNA-cross-link Repair genes, including FANCA, FANCB, FANCC; DNA-repair checkpoint genes, including ATM, ATR and p53.

Other genes include those encoding DNA polymerases. There are two classes of polymerase that may be involved in DNA repair 1) those that readthrough errors, allowing them to remain and 2) those that proof-read.

Among those that proof-read are polymerases that are involved in Translesion Synthesis, including DNA-pol η, ι and κ. In an embodiment of the invention where the DNA repair gene is a polymerase involved in translesion synthesis, there is provided an activator of that DNA repair gene and/or the proteins they encode.

For those polymerases that proof read, including, for examples POLE, POLD and POLQ, the present invention provides an inactivator of these genes and/or the proteins they encode.

Genes that may be involved in DNA repair mechanisms are listed in the following Table 1:

| Gene | |
|---|---|
| Symbol | GENEID |
| ABL1 | 25 |
| ALKBH1 | 8846 |
| ALKBH2 | 121642 |
| ALKBH3 | 221120 |
| APEX1 | 328 |
| APEX2 | 27301 |
| APLF | 200558 |
| APTX | 54840 |
| ASF1A | 25842 |
| ATF2 | 1386 |
| ATM | 472 |
| ATR | 545 |
| ATRIP | 84126 |
| ATRX | 546 |
| ATXN3 | 4287 |
| BAZ1B | 9031 |
| BLM | 641 |
| BRCA1 | 672 |
| BRCA2 | 675 |
| BRCC3 | 79184 |
| BRE | 9577 |
| BRIP1 | 83990 |
| BTG2 | 7832 |
| C7ORF11 | 136647 |
| CCNH | 902 |
| CCNO | 10309 |
| CDK7 | 1022 |
| CDKN2D | 1032 |
| CETN2 | 1069 |
| CHAF1A | 10036 |
| CHEK1 | 1111 |
| CHEK2 | 11200 |
| CIB1 | 10519 |
| CLK2 | 1196 |
| CNOT7 | 29883 |
| CSNK1D | 1453 |
| CSNK1E | 1454 |
| DCLRE1A | 9937 |
| DCLRE1B | 64858 |
| DCLRE1C | 64421 |
| DDB1 | 1642 |
| DDB2 | 1643 |
| DDX11 | 1663 |
| DLGAP5 | 9787 |
| DMC1 | 11144 |
| DNA2 | 1763 |
| DNMT1 | 1786 |
| DUT | 1854 |
| EME1 | 146956 |
| EME2 | 197342 |
| ERCC1 | 2067 |
| ERCC2 | 2068 |
| ERCC3 | 2071 |
| ERCC4 | 2072 |
| ERCC5 | 2073 |
| ERCC6 | 2074 |
| ERCC8 | 1161 |
| EYA1 | 2138 |
| EYA3 | 2140 |
| FAAP24 | 91442 |
| FAM175A | 84142 |
| FAN1 | 22909 |
| FANCA | 2175 |
| FANCB | 2187 |
| FANCC | 2176 |
| FANCD2 | 2177 |
| FANCE | 2178 |
| FANCF | 2188 |
| FANCG | 2189 |
| FANCI | 55215 |
| FANCL | 55120 |
| FANCM | 57697 |
| FEN1 | 2237 |
| FRAP1 | 2475 |
| GADD45A | 1647 |
| GADD45G | 10912 |
| GEN1 | 348654 |
| GIYD1 | 548593 |
| GTF2H1 | 2965 |
| GTF2H2 | 2966 |
| GTF2H3 | 2967 |
| GTF2H4 | 2968 |
| GTF2H5 | 404672 |
| H2AFX | 3014 |
| HEL308 | 113510 |
| HMGB1 | 3146 |
| HMGB2 | 3148 |
| HUS1 | 3364 |
| IGHMBP2 | 3508 |
| IHPK3 | 117283 |
| KAT2A | 2648 |
| KAT5 | 10524 |
| LIG1 | 3978 |
| LIG3 | 3980 |
| LIG4 | 3981 |
| MAD2L2 | 10459 |
| MBD4 | 8930 |
| MDC1 | 9656 |
| MEN1 | 4221 |
| MGMT | 4255 |
| MIZF | 25988 |
| MLH1 | 4292 |
| MLH3 | 27030 |
| MMS19 | 64210 |
| MNAT1 | 4331 |
| MPG | 4350 |
| MRE11A | 4361 |
| MSH2 | 4436 |
| MSH3 | 4437 |
| MSH4 | 4438 |
| MSH5 | 4439 |
| MSH6 | 2956 |
| MUS81 | 80198 |
| MUTYH | 4595 |
| NABP1 | 64859 |
| NABP2 | 79035 |
| NBN | 4683 |
| NEIL1 | 79661 |
| NEIL2 | 252969 |
| NEIL3 | 55247 |
| NHEJ1 | 79840 |
| NPM1 | 4869 |
| NTHL1 | 4913 |
| NUDT1 | 4521 |
| OGG1 | 4968 |
| PALB2 | 79728 |
| PARG | 8505 |
| PARP1 | 142 |
| PARP2 | 10038 |
| PARP3 | 10039 |
| PCNA | 5111 |
| PER1 | 5187 |
| PMS1 | 5378 |
| PMS2 | 5395 |
| PMS2L5 | 5383 |
| PNKP | 11284 |
| POLA1 | 5422 |
| POLB | 5423 |
| POLD1 | 5424 |
| POLE | 5426 |
| POLE2 | 5427 |
| POLG | 5428 |
| POLG2 | 11232 |
| POLH | 5429 |
| POLI | 11201 |
| POLK | 51426 |
| POLL | 27343 |
| POLM | 27434 |
| POLN | 353497 |
| POLQ | 10721 |
| POLS | 11044 |
| PRKCG | 5582 |
| PRKDC | 5591 |
| PRMT6 | 55170 |
| PRPF19 | 27339 |
| RAD1 | 5810 |
| RAD17 | 5884 |
| RAD18 | 56852 |
| RAD21 | 5885 |
| RAD23A | 5886 |
| RAD23B | 5887 |
| RAD50 | 10111 |
| RAD51 | 5888 |
| RAD51C | 5889 |
| RAD51L1 | 5890 |
| RAD51L3 | 5892 |
| RAD52 | 5893 |
| RAD54B | 25788 |
| RAD54L | 8438 |
| RAD9A | 5883 |
| RASSF7 | 8045 |
| RBBP8 | 5932 |
| RDM1 | 201299 |
| RECQL | 5965 |
| RECQL4 | 9401 |
| RECQL5 | 9400 |
| REV1 | 51455 |
| REV3L | 5980 |
| RNF168 | 165918 |
| RNF8 | 9025 |
| RPA1 | 6117 |
| RPA2 | 6118 |
| RPA3 | 6119 |
| RPA4 | 29935 |
| RPAIN | 84268 |
| RPS27L | 51065 |
| RRM2 | 6241 |
| RRM2B | 50484 |
| RTEL1 | 51750 |
| RUVBL2 | 10856 |
| SETMAR | 6419 |
| SETX | 23064 |
| SHFM1 | 7979 |
| SIRT1 | 23411 |
| SMC1A | 8243 |
| SMC3 | 9126 |
| SMC6 | 79677 |
| SMUG1 | 23583 |
| SOD1 | 6647 |
| SPO11 | 23626 |
| TADA3L | 10474 |
| TCEA1 | 6917 |
| TDG | 6996 |
| TDP1 | 55775 |
| TDP2 | 51567 |
| TNP1 | 7141 |
| TOP2A | 7153 |
| TOPBP1 | 11073 |
| TP53 | 7157 |
| TP53BP1 | 7158 |
| TP73 | 7161 |
| TREX1 | 11277 |
| TREX2 | 11219 |
| TRIM28 | 10155 |
| TRIP13 | 9319 |
| TYMS | 7298 |
| UBE2A | 7319 |
| UBE2B | 7320 |
| UBE2N | 7334 |
| UBE2V1 | 7335 |
| UBE2V2 | 7336 |
| UIMC1 | 51720 |
| UNG | 7374 |
| UPF1 | 5976 |
| USP1 | 7398 |
| UVRAG | 7405 |
| VCP | 7415 |
| WRN | 7486 |
| XAB2 | 56949 |
| XPA | 7507 |
| XPC | 7508 |
| XRCC1 | 7515 |
| XRCC2 | 7516 |
| XRCC3 | 7517 |
| XRCC4 | 7518 |
| XRCC5 | 7520 |
| XRCC6 | 2547 |
| XRCC6BP1 | 91419 |
| YBX1 | 4904 |

In one embodiment of the invention a DNA repair gene is an MMR gene i.e. a gene whose protein product is involved in mismatch repair (MMR). Suitable genes include MLH1, MSH2 and PMS2.

In one embodiment, a DNA repair gene may be MSH3, MSH6, ATR, RAD50, POLE, POLO, FANCM, ATM, PRKDC, POLQ or DNMT1.

Suitably, modifying a DNA repair gene/protein to inactivate it results in the DNA mutation (and therefore the corresponding neo-antigen) profiles dynamically evolving over time. In one embodiment, the modification leads to the generation of neo-antigens (tumour antigens). In one embodiment, the invention relates to increase of ‘dynamic’ mutational loads that can be achieved by inactivation of DNA repair genes/proteins.

Modification of a DNA repair gene may be measured at number of different levels. In one embodiment, a functional assay may be performed to analyse e.g. the ability of a test compound to bind to a protein encoded by a DNA repair gene and/or the ability of that test compound to inhibit the function of that gene. Suitable assays for particular types of proteins involved in DNA repair will be familiar to those skilled in the art. For example, assays for non-homologous end joining (NHEJ), Microhomology mediated end joining (MMEJ), Homologous Recombination (HR), Mismatch repair (MMR), Base Excision Repair (BER), Nucleotide Excision Repair (NER), DNA-cross-link Repair, DNA-repair checkpoint and DNA polymerases are available.

In another embodiment, modification of a DNA repair gene may be determined by measuring DNA mutations and, in particular by measuring the rate of mutation. Suitable methods for determining an accumulation of mutations or rate of mutation are described herein. In one embodiment, increased mutation rates may be determined by measuring the number of neo antigens.

Suitably in a treatment in accordance with the invention, a modifier of a DNA repair gene or protein is provided in a therapeutically effective amount. The term “therapeutically effective amount” refers to that amount of the compound being administered which will relieve to some extent one or more of the symptoms of the disease of disorder being treated. In one embodiment, the treatment may be relatively prolonged, e.g. over a number of months.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Prior to administration of a modifier as part of a treatment in accordance with the invention, a patient may be screened to determine whether a cancer from which the patient is or may be suffering is one which is characterised by the presence of an active form of a DNA repair gene or enzyme. For example, a cancer may be identified as an MMR +ve cancer i.e. a cancer in which those genes involved in MMR are active and/or present or have not been lost as part of the tumour evolution. Presence of genes involved in a DNA repair process such as MMR may be detected using methods familiar to those skilled in the art and include, for example, PCR methods.

The phrase “manufacture of a medicament” includes the above described compound directly as the medicament in addition to its use in a screening programme for further active agents or in any stage of the manufacture of such a medicament.

### Nucleic Acid Editing Mechanisms

In one embodiment of any aspect of the invention, the present invention provides for modifying genes such as those involved in nucleic acid editing. Suitably, the invention may relate to any genes whose modulation leads to an increase in rates or loads of DNA mutations, or epi-mutations at the level of RNA.

In addition to extrinsic factors such as radiation or chemical mutagens that induce altered expression of protein products that encode mutant variants of germline-encoded genes, there are a variety of intrinsic mechanisms that reversibly or irreversibly alter the coding protein complement. In the endogenous process of nucleic acid editing, certain nucleotide bases undergo conversion to alternate bases following enzyme activity. Genes that may be involved in nucleic acid editing mechanisms are listed in the following Table 2:

| Gene Symbol | GENE ID |
|---|---|
| ADAR | 103 |
| ADARB1 | 104 |
| ADARB2 | 105 |
| ADARB2-AS1 | 642394 |
| APOBEC3D | 140564 |
| APOBEC3B | 9582 |
| APOBEC3C | 27350 |
| APOBEC3G | 60489 |
| APOBEC3F | 200316 |
| APOBEC3A | 200315 |
| APOBEC3H | 164668 |
| APOBEC1 | 339 |
| A1CF | 29974 |
| APOBEC2 | 10930 |
| APOBEC4 | 403314 |
| APOBEC3AP1 | 105377532 |
| APOBEC3B-AS1 | 100874530 |

In one embodiment of the invention, a nucleic acid editing gene is the activation induced cytidine deaminase gene (AID or AICDA), which is employed by somatic cells of the immune system to irreversibly modify the coding composition and diversity of immunoglobulin genes, by inducing Igg somatic hypermutation and class-switch recombination. While the endogenous AICDA gene and its homologs are employed to broaden the genetic diversity of healthy somatic cells, the activity of this gene can also introduce carcinogenic somatic mutations in B-cell malignancies, including some which appear to present novel antigens.

A variety of additional nucleic acid editing enzymes appear to influence both natural and carcinogenic DNA alterations. In another embodiment of the invention, a nucleic acid editing gene is a member of the apolipoprotein-B editing cytidine deaminase (APOBEC) gene family; these genes are employed by normal cells to edit and covert cytidine bases in messenger RNAs to uracil, which induces novel post-transcriptional isoforms of natural genes. Additionally, enzyme activity of the APOBEC family appears to play an ancestral role in silencing and restricting the activity of both human viruses and endogenous retroviruses. As with the AICDA gene, APOBEC enzyme activity seems to similarly influence carcinogenic DNA mutations: a major subset of human cancers exhibit mutation patterns consistent with elevated and spurious APOBEC activity. Here, it is likely that APOBEC enzymes modify the bases of single-stranded DNA ends that are intermittently present during DNA replication and DNA damage. As such, a variety of human cancers and human cancer antigens are likely caused by excessive APOBEC enzyme activity.

In another embodiment of the invention, a nucleic acid editing enzyme is one of the RNA-editing adenosine deaminase enzyme family (ADARs) genes, which also appear to play a role in both normal and carcinogenic post-transcriptional alterations in protein expression. Here again, the natural function of ADAR genes in dsRNA virus response appears to be subverted in several instances, where enzyme activity introduces adenosine to inosine base alterations that change the coding potential of endogenous mRNAs. Altered ADAR enzyme activity and consequent changes in mRNA content beyond its germline DNA coding configuration is observable in both cancer cells like hepatocellular carcinoma, as well as in autoimmune disorders.

Other molecules which may edit nucleic acids include splicing factors. ADATs for tRNA modifications may also be envisaged.

Suitably, modification of nucleic add editing mechanisms will result in the DNA mutation or RNA epimutation profiles dynamically evolving over time, and therefore correspondingly alter the expression and presentation of neo-antigens to the immune system. In one embodiment, the modification leads to the generation of neo-antigens (tumour antigens). In one embodiment, the invention relates to increase of ‘dynamic’ mutational loads that can be achieved by inactivation of DNA repair genes/proteins.

In another embodiment, modification of a nucleic acid editing gene may be determined by measuring DNA or RNA mutations and, in particular by measuring the rate of mutation. Such mutations may include point mutations, frameshift mutations and mutations as a result of homologous recombination. Suitable methods for determining an accumulation of mutations or rate of mutation are described herein. In one embodiment, increased mutation rates may be determined by measuring the number of neo antigens.

Suitably in a treatment in accordance with the invention, a modifier of a nucleic acid editing gene or protein is provided in a therapeutically effective amount. The term “therapeutically effective amount” refers to that amount of the compound being administered which will relieve to some extent one or more of the symptoms of the disease of disorder being treated. In one embodiment, the treatment may be relatively prolonged, e.g. over a number of months.

Herein, the term “treating” includes abrogating, substantially inhibiting, slowing or reversing the progression of a disease or disorder, substantially ameliorating clinical symptoms of a disease or disorder or substantially preventing the appearance of clinical symptoms of a disease or disorder.

Prior to administration of a modifier as part of a treatment in accordance with the invention, a patient may be screened to determine whether a cancer from which the patient is or may be suffering is one which is characterised by the presence of an altered form of a nucleic acid editing gene or enzyme. For example, a cancer may be identified as an cancer that exhibits AICDA-APOBEC dependent or “kataegis” DNA hypermutation i.e. a cancer in which those genes involved in nucleic acid enzyme AICDA or APOBEC(s) have been altered as part of tumour evolution. Presence of genes involved in a nucleic acid editing process may be detected using methods familiar to those skilled in the art and include, for example, PCR methods.

The phrase “manufacture of a medicament” includes the above described compound directly as the medicament in addition to its use in a screening programme for further active agents or in any stage of the manufacture of such a medicament.

### Test Compound

A test compound for use in an assay in accordance with any aspect of any embodiment of the invention may be a protein or polypeptide, polynucleotide, antibody, peptide or small molecule compound. In one embodiment, the assay may encompass screening a library of test compounds e.g. a library of proteins, polypeptides, polynucleotides, antibodies, peptides or small molecule compounds. Test compounds may also comprise nucleic acid constructs such as CRISPR constructs, siRNA molecules, anti-sense nucleic acid molecules and so forth. Suitable high throughput screening methods will be known to those skilled in the art.

Other methods for identifying a suitable test compound that may be used as a modifier of a DNA repair or nucleic acid editing gene or protein in accordance with any aspect or embodiment of the invention include the rational design of compounds. In this approach, a compound library for screening may be based on starting with those compounds known to bind to and/or inhibit/inactivate or to enhance/activate a molecule having structural similarity or homology to the DNA repair or nucleic acid editing gene of interest.

For example, a structural analysis of MLH1 suggests that it shares structural homology with the bacterial enzyme, DNA gyrase. Thus a rational drug design approach may start with known inhibitors of DNA gyrase as a basis for deriving a compound library for testing in a screening method in accordance with the present invention. Suitable starting points for this method are described, for example, by Collin et al., Appl Microbiol Biotechnol (2011) 92: 479-497.

### Combinations

In one embodiment, of any aspect of the invention, a treatment using a compound which acts to modify e.g. activate or inactivate a DNA repair or nucleic acid editing gene or protein may be provided as a therapy alone, for example as a monotherapy. In other embodiments, the compound which acts to modify a DNA or nucleic acid editing repair gene may be used in combination with another, different cancer therapy. Thus, an individual with cancer may be given an initial treatment such as chemotherapy, with a compound that acts to modify a DNA repair or nucleic acid editing gene being administered so as to be effective in the rapid resistance outgrowth phase post treatment. In particular, the present invention includes combinations of modifiers of DNA repair or nucleic acid editing genes with immune checkpoint inhibitors.

As described herein, chemotherapeutic agent or natural products that may be effective to select for cancer cells in which MMR genes will have been inactivated e.g., MNNG, 6TG, Temozolomide.

### Types of Cancer

Examples of cancers (and their benign counterparts) which may be treated include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the oesophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukaemias, lymphomas) and premalignant haematological disorders and disorders of borderline malignancy including haematological malignancies and related conditions of lymphoid lineage (for example acute lymphocytic leukaemia [ALL], chronic lymphocytic leukaemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukaemia [AML], chronic myelogenous leukaemia [CML], chronic myelomonocytic leukaemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukaemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcoma protuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

In one embodiment, a tumour for treatment in accordance with the invention may be one which has a mutation in a DNA repair or nucleic acid editing gene.

For example, Hereditary Non Polyposis Colon Cancer (HNPCC) is a hereditary cancer syndrome comprising a germline mutation in genes controlling MMR. Accordingly, HNPCC is one cancer syndrome that may be treated in accordance with the invention or using a compound identified using a method of screening in accordance with the invention. Other suitable cancers having mutations in DNA repair genes will be familiar to those skilled in the art.

Other cancers that have alterations in MMR genes are described, for example, in Xiao et al. (2014) and Okuda et al. (2010), and include sporadic colorectal cancers, ovarian and endometrial cancers.

In one aspect, the invention also provides a method for selecting those individuals most likely to respond to an immunotherapeutic approach by identifying patients having defects in DNA repair or nucleic acid editing mechanisms. Thus, a patient may be screened to determine whether a cancer from which the patient is or may be suffering is one which is characterised by elevated levels of DNA mutation and which would therefore be would be susceptible to treatment with a compound having an immunomodulatory approach such as those immune check point inhibitors.

For example, a diagnostic test may be undertaken. Suitably, a biological sample taken from a patient may be analysed to determine whether a cancer, that the patient is or may be suffering from, is one which is characterised by a genetic abnormality or abnormal protein expression which leads to an increased mutation rate. An increased mutation rate may be determined by measuring the number of mutations over time, for example, using a microsatellite analysis as described herein.

The diagnostic tests are typically conducted on a biological sample selected from tumour biopsy samples, blood samples (isolation and enrichment of shed tumour cells), stool biopsies, sputum, chromosome analysis, pleural fluid and peritoneal fluid.

Other aspects and embodiments of the invention are set out in the following clauses:
1. A method for treating cancer comprising:
   a) providing i) a subject having cancerous cells, and ii) a modifier of a DNA repair gene or its protein product; and
   b) treating said subject with said modifier wherein said treating reduces the number of cancerous cells in said subject.
2. A method according to clause 1 wherein the modifier of a DNA repair gene or its protein product is an activator.
3. A method according to clause 2 wherein the DNA repair gene encodes a protein involved in translesion synthesis.
4. A method according to clause 3 wherein the DNA repair gene is DNA-pol η, ι or κ.
5. A method according to clause 1 wherein the modifier of a DNA repair gene is an inactivator.
6. A method according to clause 7 wherein the DNA repair gene is an MMR gene.
7. A method according to clause 8 wherein the MMR gene is a MutL homologue.
8. A method according to clause 9 wherein the MutL homologue is MLH1, MutLα, MutLβ, MutLγ, PMS1, PMS2 or MLH3.
9. A method according to any preceding clause wherein the modifier is a polypeptide, polynucleotide, antibody, peptide or small molecule compound.
10. A method according to any preceding clause wherein an inactivator is a molecule which provides inactivation through genome editing.
11. A method according to any preceding clause wherein the cancer is an MMR +ve cancer.
12. A method for treating cancer according to any preceding clause wherein the modifier of a DNA repair enzyme is provided in combination with a different cancer treatment.
13. A method according to clause 12 wherein the different cancer treatment is one which inactivates MMR genes.
14. A method according to clause 13 wherein the different cancer treatment is treatment with temozolomide or MNU or their derivatives.
15. A method according to clause 15 wherein the different cancer treatment is an immunotherapy.
16. A method according to clause 18 wherein the immunotherapy is an immune checkpoint inhibitor or combination of immune checkpoint inhibitors.
17. A method according to clause 19 wherein the immune checkpoint inhibitor is an anti-PD1 antibody, an anti-CTLA-4 antibody, an anti-PDL-1 antibody or combinations thereof.
18. A method of treatment according to any preceding clause wherein the inactivator/inhibitor of a mismatch repair gene is temozolomide, MNU or their derivatives.
19. A method for screening for anti-cancer compounds comprising
   a) providing cells expressing a DNA repair gene;
   b) incubating said cells in the presence of a test compound;
   c) measuring the rate of DNA mutation in the presence of a test compound;
   d) wherein an increased rate of DNA mutation in cells in the presence of a test compound compared to that measured in cells in the absence of a test compound indicates a test compound is an anti-cancer compound.
20. A method according to clause 22 wherein cells expressing a DNA repair gene are a human tumour cell line.
21. A method according to clause 22 or clause 23 wherein the DNA repair gene is MLH1, MutLα, MutLβ, MutLγ, PMS1, PMS2 or MLH3.
22. A method of identifying a patient having a tumour suitable for treatment by immunotherapy comprising:
   a) taking a sample of said tumour,
   b) analysing said sample to determine the sequence of a DNA repair gene
   c) comparing the sequence of said DNA repair gene in a tumour sample with the sequence in a non-tumour sample
   wherein a defect in the sequence of said DNA repair gene in the tumour sample compared to the sequence of said gene in a non-tumour sample is indicative that said patient has a tumour suitable for treatment by immunotherapy.

Certain aspects and embodiments of the invention will now be illustrated by way of example and with reference to the following Figures and Examples.

### FIGURES

FIG. 1. In vivo consequences of MLH-1-inactivation in CT26, colorectal cancer and TS/A breast cancer cell lines.

CRISPR/CAS9 mediated knock-out of MLH-1 was obtained in CT26 colorectal cell lines (A). CT26 were infected and, after puromycin selection, single cell cloning was performed. CTRL represents a CT26 clone infected with CRIPR/CAS9 vector without guide. M2 and M3 were two different clones obtained with guide number 2 and 3 respectively. Two guides were chosen in order to avoid any off-target effect. CT26 clones were injected (5×10⁵ cells per mouse) subcutaneously in NOD/SCID mice and the growth was monitored until the day of sacrifice. (B) CT26 clones were injected in BalbC immunocompetent mice. The majority of M2 and M3 clones were rejected (11 out of 14 and 10 out of 14 respectively) showing that the same cells that grew in immune-deficient mice were rejected once in immune-competent system. (C) Survival curve of BalbC mice injected in (B) with control (solid line), M2 (black dotted line) and M3 (black small dotted line). MLH-1 KO guaranteed the survival of the majority of injected mice for more than three months (D) MLH-1 deficient cells were obtained also from TS/A breast cancer cell lines. For TS/A we selected two MLH-1 KO clones. M3 and M6 represent two clones from guide number 3 and 6 respectively. The western blot showed the MLH-1 level after three months of in vitro culture. MLH-1 KO and WT clones were injected in immune-deficient mice. (E) MLH-1 proficient and deficient cells where injected (5×10⁵ cells per mouse) subcutaneously in BalbC mice. The majority of mice with MLH-1 KO clones rejected (6 out of 7 mice) whereas the CTRL grew. Statistical analysis: *p<0.05, **p<0.01, ***p<0.001 (Student's t test). (F) Survival curve of TS/A was obtained from mice of experiment in (E).

FIG. 2. MLH-1 inactivation in pancreatic cancer cell lines confers immunogenic rejection.

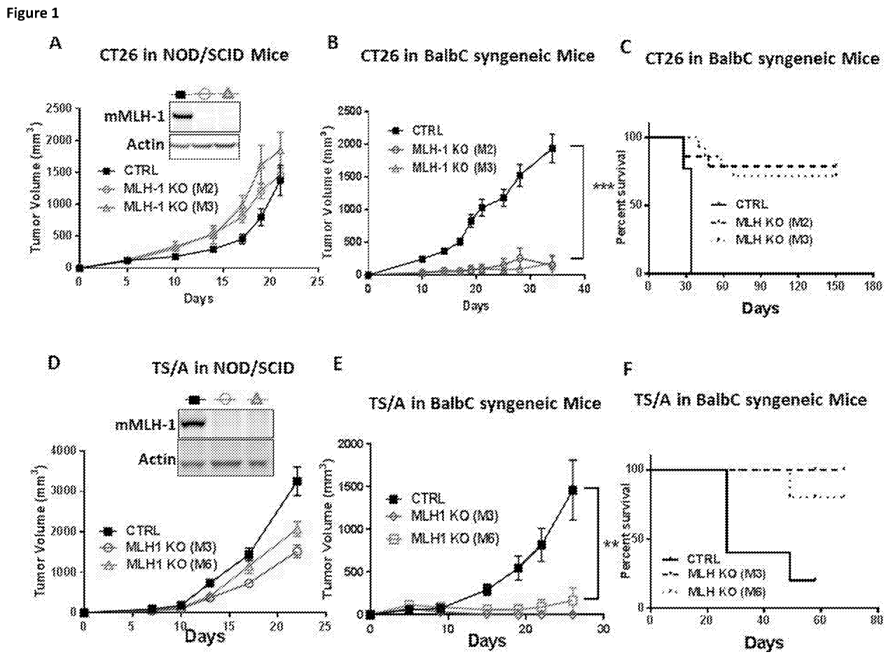

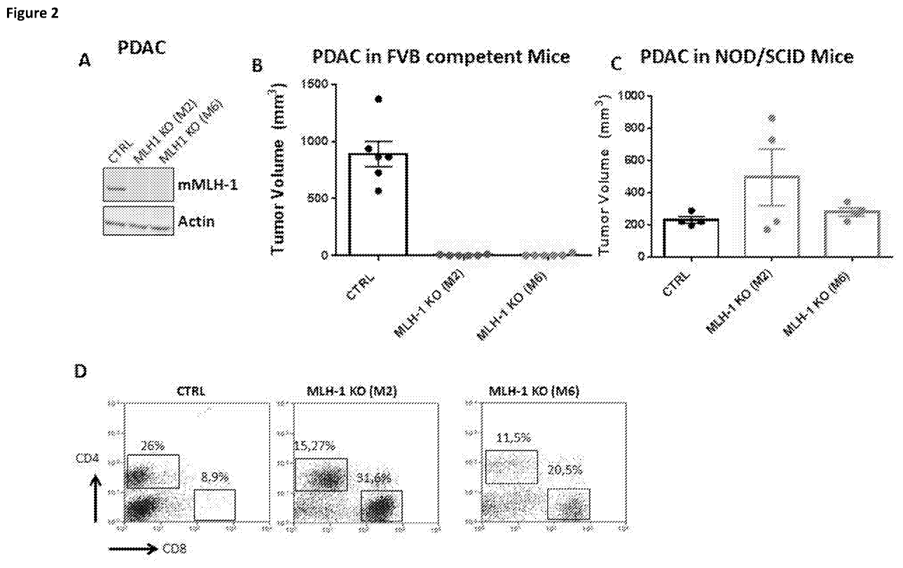

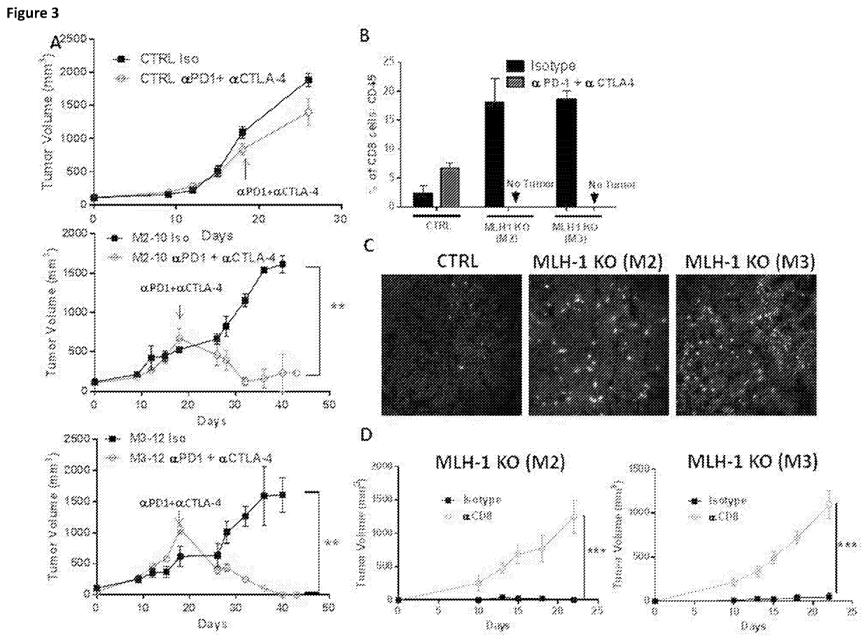

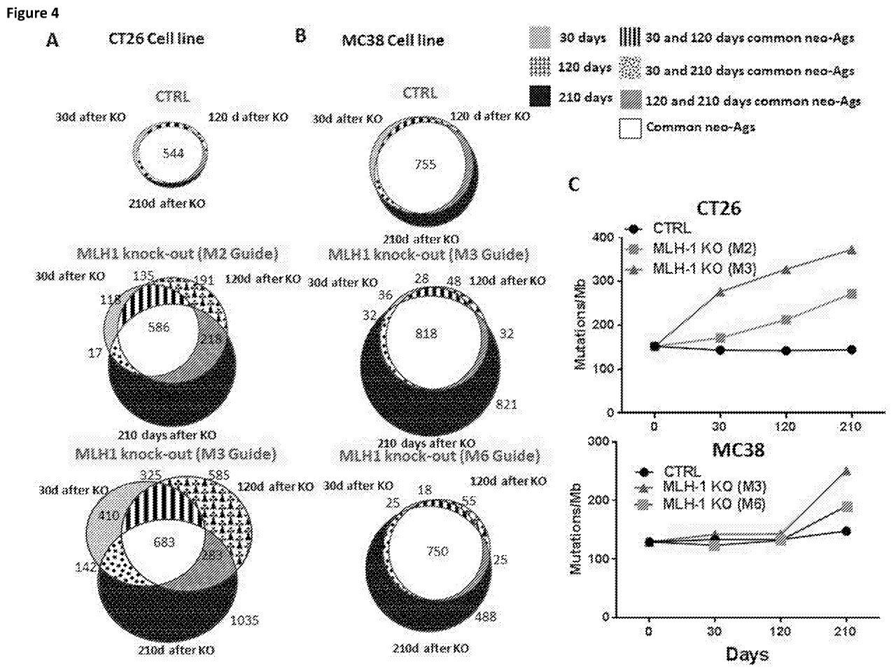

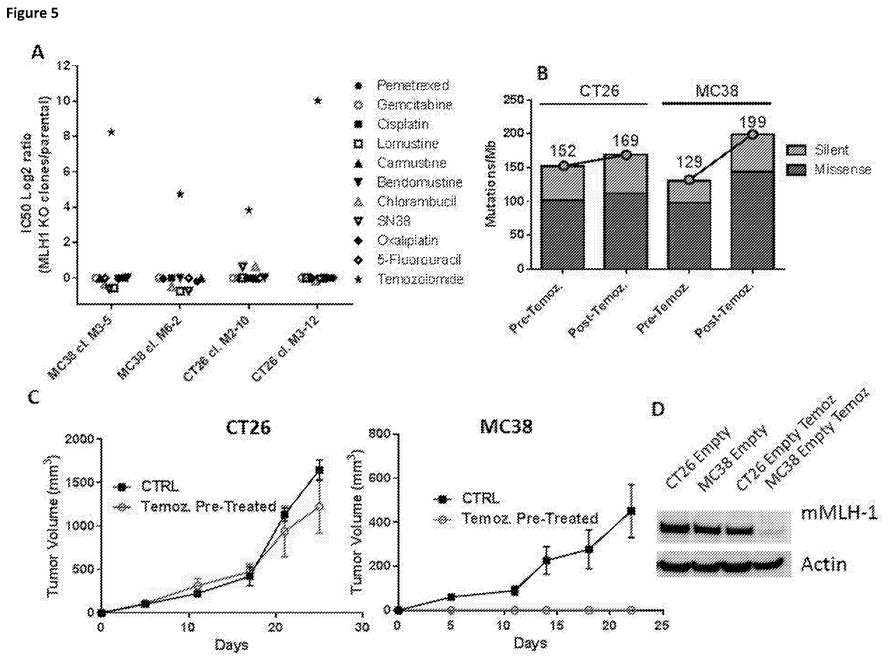

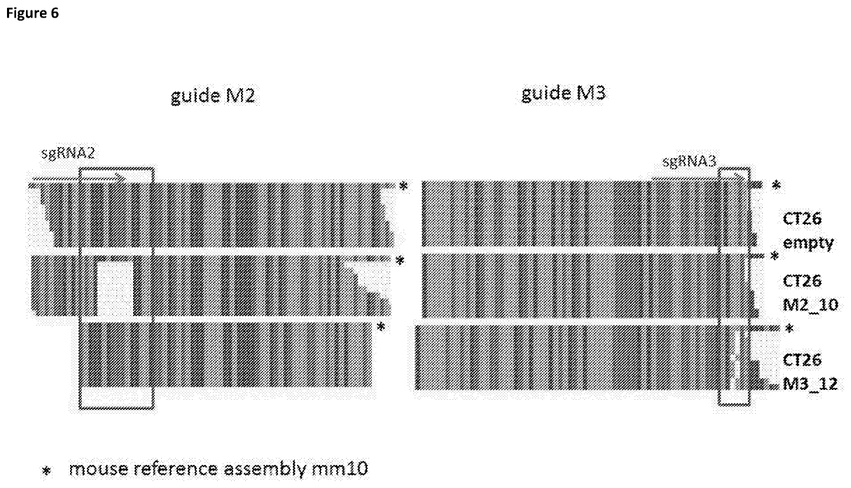

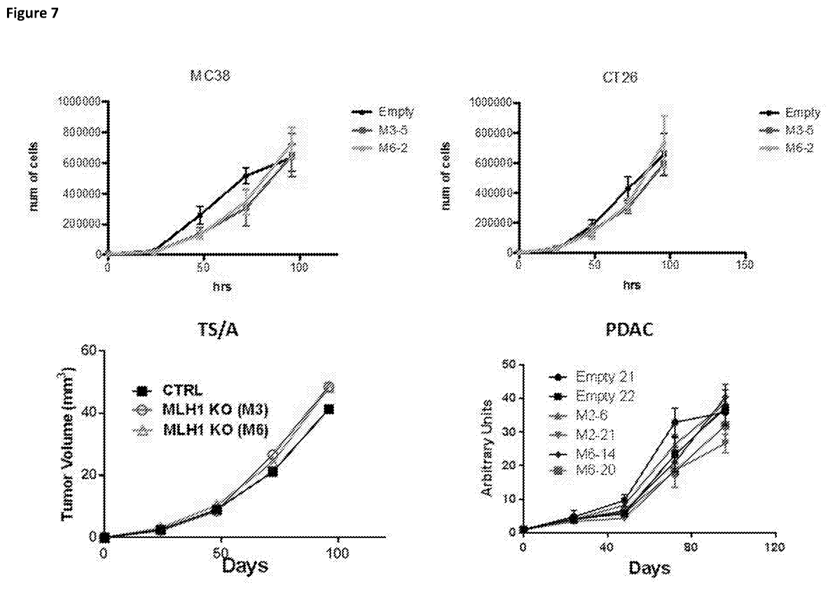

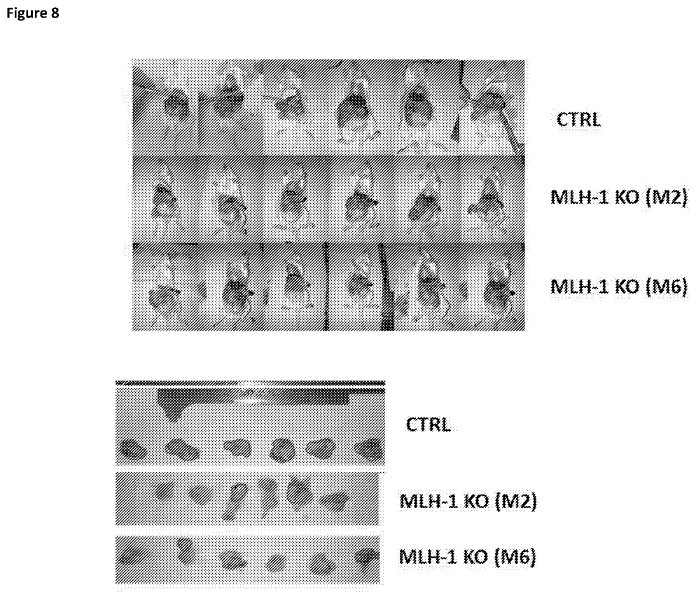

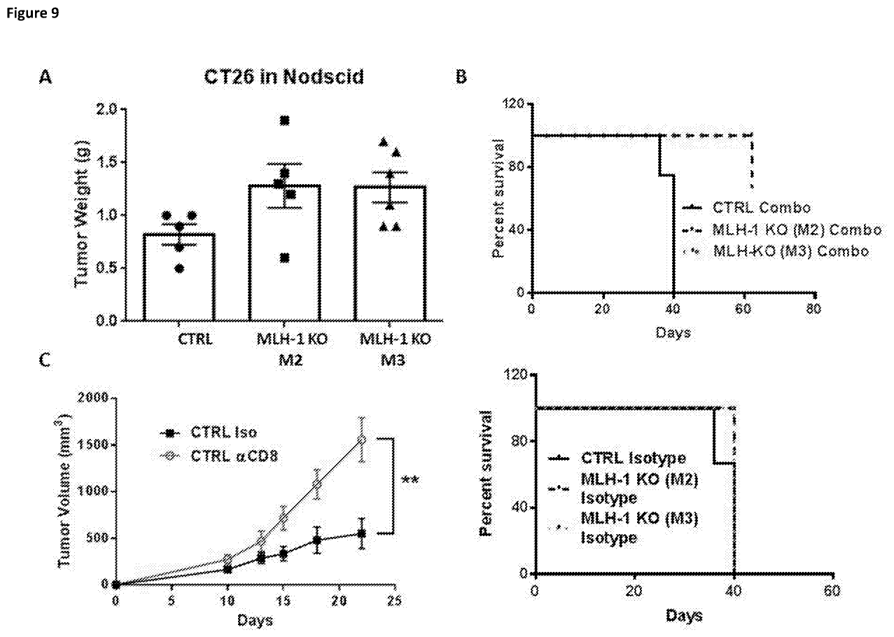

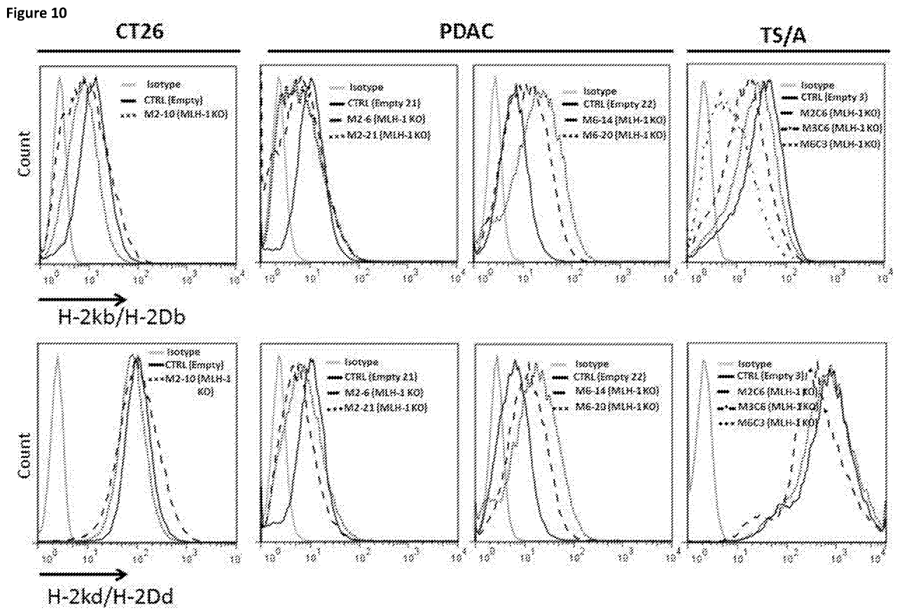

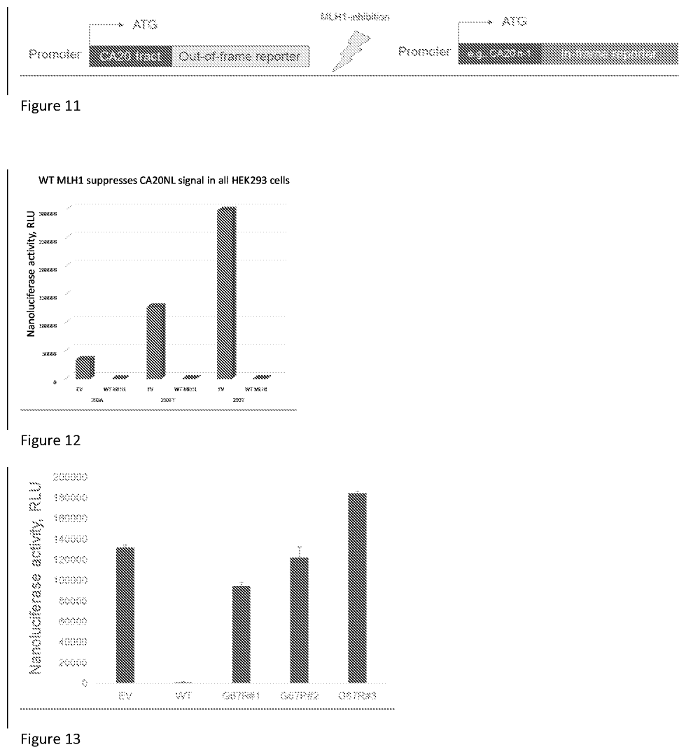

(A) The same approach followed in FIG. 1 was followed for PDAC cell lines. MLH-1 was monitored for four months in two control clones and in MLH-1 KO clones infected with guide 2 and 6. (B) MLH-1 proficient and deficient clones were injected in NOD/SCID mice where they showed an intrinsic ability for the engraftment in mice without a proficient immune system. (C) PDAC cells were injected (10³ cells per mouse) orthotopically in FVB mice. After three weeks mice were sacrificed and tumour burden was measured. Tumour volume of pancreatic tumour showed that MLH-1 silencing interfered with tumour growth. (D) Pancreatic tumour mass was dissociated analyzing the percentage of CD8 and CD4 T cells. Statistical analysis: *p<0.05, **p<0.01, ***p<0.001 (Student's t test).

FIG. 3. MLH-1 inactivation made CT26 responsive to anti-PD-1 and anti-CTLA-4 therapy in BalbC mice.

(A) Tumours that grew in NOD/SCID mice were dissected into pieces of 2 mm per side and implanted subcutaneously at the level of the left leg. After 18 days anti-PD1 (250 μg per mouse) and anti-CTLA4 (200 μg per mouse) were administrated i.p. for three times every three days. After that therapy continued with anti PD-1 every three days. Tumours in MLH1 KO mice responded to anti PD-1 and anti CTLA-4 therapy whereas tumours with proficient MLH1 showed only a delay in growth. (B) Percentage of CD8 T cells was higher in MLH deficient tumour compared to control. In the MLH-1 proficient tumour, therapy increased the percentage of CD8 T cells in the tumour. Statistical analysis: *p<0.05, **p<0.01, ***p<0.001 (Student's t test). (C) CD8 infiltration was clearly higher in MLH1 KO compared to MLH-1 proficient tumour owing to the increased level of neo-antigens in MLH-1 KO clones. (D) CT26 were injected (5×10⁵ cells per mouse) and the same day mice were treated with anti CD8 depleting antibody (400 μg per mouse at day 0, 100 μg per mouse at day 1 and 100 μg at day 2). Depletion of CD8 T cells in MLH-1 KO tumours guaranteed tumour growth confirming that those cells controlled tumour escape in MLH1 deficient tumours.

FIG. 4. Mutational load over-time on CT26 and neo-antigen prediction.

(A) CT26 clones were compared over time in order to track the evolution of suitable neo-antigens. Variants reported in the annotation file were used for calculating mutated peptide sequences and loaded into NetMHC 4.0 software getting out predicted neo-antigens. (B) The same was performed for MC38 cell lines. (C) Starting from the initial pool of CT26 clones, mutational burden was calculated at each timepoint for each clone. The total number of single nucleotide coding variants (germline ones kept out) if supported at least 1% allelic frequency, it was normalized on coding exome and reported in mutation rate (bases per million).

FIG. 5. Drug screening of alkylating agents in MLH-1 proficient and deficient tumours identified temozolomide as a promising therapeutic approach.

(A) Drug screening of alkylating agents on cell lines with or without MLH-1. Differential susceptibility of cells was measured throughout crystal violet staining. The amount of crystal violet was dissolved and quantified by spectrophotometer. In the figure A the IC50 of Log 2 ratio between MLH-1 KO and wt identified which drug had a lower effect on the survival of MLH-1 KO compared to parental. (B) Mutational load of CT26 and MC38 after Temozolomide treatment. Cells were treated, starting from 100 μM to 500 μM of Temozolomide, for 4 months. (C) CT26 and MC38 were injected subcutaneously (5×10⁴ cells per mouse). Tumour volume was measured twice a week. (D) MLH-1 expression in CT26 and MC38 after temozolomide treatment. The MLH-1 level was strongly reduced in MC38 whereas no differences were appreciated in CT26.

FIG. 6. MLH-1 sequence alignment in CT26 clones.

MLH-1 regions were identified according to the guide numbers 2 and 3. Clones with guide 2 showed a deletion of 8 bps that induced a frameshift of 22 codons. Guide 3 produced a frameshift of 9 codons owing to a double deletion. The first line is the mouse reference assembly mm10.

FIG. 7. In vitro growth of CT26, MC38, TS/A and PDAC cell lines.

Colorectal cancer cell lines were plated 1000 cells per well. TS/A and PDAC were plated at 5000 cells per well. All clones of MLH-1 KO were tested for in vitro growth. Their metabolic activity was quantified with Cell Titer Glo every 24 hours.

FIG. 8. PDAC tumour-bearing mice at the day of sacrifice.

PDAC tumour bearing mice at the day of sacrifice. Picture showed the size of tumour in all clones analyzed.

FIG. 9.

Effects of immune control on CT26 clones in vivo.

(A) CT26 clones injected in immune-deficient mice were explanted. The weight of tumours showed an increased size for MLH-1 KO clones however not reaching statistical significance. (B) Survival curve of mice transplanted with CT26 showed that the absence of MLH-1 made tumours more responsive to immune-checkpoint inhibitors (B upper panel). Isotype control mice and MLH-1 KO tumours were sacrificed the same days for ethical reasons. (C) CD8 T cells were involved in tumour rejection in MLH-1 KO CT26. The absence of CD8 T cells enhanced tumour growth also in MLH-1 proficient tumour. The absence of CD8-mediated control increased the growth rate of CT26 control cells.

FIG. 10. Expression of MHCI in CT26, PDAC and TS/A murine cell lines.

Cell lines were stained with anti-H-2 kb/H-2Db and H-2kd/H-2Dd PE labelled antibody. Cells were harvested and after a single wash in FACS Buffer (PBS+2% FBS), were incubated for 30 min at 4° C. with antibodies. The analysis revealed that all clones used were MHCI proficient.

FIG. 11. CA₍₂₀₎ NanoLuc assay

In the presence of proficient post-replicative MMR, the reporter gene remains out of frame. Inhibited or genetically deficient MMR leads to frameshift mutations and an in-frame reporter which is detected using commercial NanoLuciferase assay systems.

FIG. 12.

Cells were transfected with CA₍₂₀₎-NanoLuc plasmid and either a control empty vector (EV) or a plasmid expressing wild-type (WT) MLH1. 24 hours after transfection, cells were trypsinised, counted and replated into 96-well plates at 10,000 cells per well in 8 replicates per condition. Cells were then cultured for 72 hours and then NanoLuciferase reporter activity was detected using the NanoGlo assay system (Promega) from 4 of the wells. Luminescence was measured on a BMG Clariostar plate reader. Cell number was normalised for using the Cell Titre Blue reagent (Promega), also read on the Clariostar, from the remaining 4 wells. Data is shown as normalised NanoLuciferase activity normalised to the Cell Titre Blue data.

FIG. 13.

Cells were transfected with CA₍₂₀₎-NanoLuc plasmid and either a control empty vector (EV) a plasmid expressing wild-type (WT) MLH1, or MLH1 G67R clones #1, 2 or 3. 24 hours after transfection, cells were trypsinised, counted and replated into 96-well plates at 10,000 cells per well in 8 replicates per condition. Cells were then cultured for 72 hours and then NanoLuciferase reporter activity was detected using the NanoGlo assay system (Promega) from 4 of the wells. Luminescence was measured on a BMG Clariostar plate reader. Cell number was normalised for using the Cell Titre Blue reagent (Promega), also read on the Clariostar, from the remaining 4 wells. Data is shown as normalised NanoLuciferase activity normalised to the Cell Titre Blue data.

### EXAMPLES

### Example 1

### Summary

Molecular alterations in tumour suppressor genes involved in DNA mismatch repair (MMR) promote cancer initiation and foster tumour progression. MMR deficient cancers frequently show favourable prognosis and indolent progression. The functional basis of the clinical outcome of patients with MMR tumours was addressed. MutL homolog 1 (MLH1) in colorectal, breast and pancreatic mouse cancer cells was genetically inactivated. MMR deficient cells grew at equal or higher rates than their proficient counterparts in vitro and when transplanted in immune-compromised mice. Strikingly however, MMR deficient colorectal and breast cancer cells were largely unable to form tumours when injected subcutaneously in syngeneic mouse models. When transplanted orthotopically, MMR proficient pancreatic cancer cells rapidly led to fatal disease, while their MMR deficient counterparts did not grow, or formed smaller tumours. MMR deficient tumours displayed high levels of infiltrating T cells and suppression of T lymphocytes allowed exponential growth of MMR deficient tumours in syngeneic mice. MMR deficient tumours initially established in immune-deficient mice grew exponentially when transplanted in syngeneic animals but regressed completely when immune checkpoint inhibitors were administered. Sequencing of MMR proficient cells revealed high mutational loads (50-100 mutations/Mb) and neo-antigen profiles that were stable over time. MMR inactivation further increased the mutation burden, and led to persistent renewal of neo-antigens. Using a pharmacological screening it was found that increasing the mutational levels per se is not sufficient to provoke immune-surveillance. On the contrary drug-induced permanent inactivation of DNA repair leads to a dynamic hyper-mutation status that is central to immune surveillance. These results provide the rationale for developing innovative anticancer therapies.

### Results

To functionally define the role of mismatch repair in tumour formation and response to therapy MMR-proficient colorectal (CT26, MC38), breast (TSA) and pancreatic (PDAC) mouse cancer cells were studied. Genome editing with the CRISPR-CAS system was employed to inactivate MutL homolog 1 (MLH1) in each of these cell models. Independent RNA guides directed against distinct MLH1 exonic regions were used and multiple clones were isolated. Clones derived from cells treated without specific RNA guides served as controls (CTR clones). Inactivation of MLH1 was confirmed at the genomic level (FIG. 6) and at the protein level (FIG. 1A, FIG. 1D, FIG. 2A). Functional inactivation of DNA mismatch repair was established by sequencing repetitive mouse DNA elements, which were selected based on homology to equivalent regions of the human genome (FIG. 6).

In vitro, the proliferative rates of MMR-deficient cells were comparable to that of their parental derivatives and of the CTR clones (FIG. 7). Colorectal and breast MMR-deficient cells rapidly developed tumours when injected subcutaneously into immune-compromised mice and in a few weeks the animals had to be sacrificed according to ethical guidelines (FIGS. 1A and 1D). MMR-deficient cells grew faster than the control although at the experimental endpoint the difference did not reach statistical significance (FIG. 9A).

When CT26 cells were injected in the corresponding syngeneic mouse models (BalbC) they grew rapidly and after 30 days the animals had to be sacrificed. On the contrary MMR deficient cells (M2 and M3 clones) did not engraft or formed small tumour masses that regressed after a few days (FIG. 1B). The entire cohort was monitored for over three months, during which there was no evidence of tumour relapse suggesting that the mice had been cured (FIG. 10).

The same type of experiment was performed using breast cancer cells in which MLH1 had been inactivated (FIG. 1E) in syngeneic mice. Also in this case MMR deficient cells did not form tumours or formed small lesions, which subsequently regressed. On the contrary MMR proficient breast cancer cells grew rapidly and lead to the sacrifice of the animals in less than a month (FIG. 1E). The survival of MMR-deficient tumour-bearing mice was longer than the control (FIG. 1F).

It is known that when cancer cells are injected subcutaneously, the structure and the properties of the tumour stromal components and of the microenvironment are not properly reconstituted.¹⁵ As a third model pancreatic ductal adenocarcinoma (PDAC) cells were injected orthotopically in the pancreas of syngeneic mice.¹⁶ This cancer model closely recapitulates the molecular features of human PDACs. Like their human counterpart, mouse PDAC cells are extremely aggressive leading to tumours which are rapidly fatal. Notably, also in this case we observed a striking difference between MMR-proficient and MMR-deficient cells (FIG. 2B and FIG. 8). Three weeks after transplantation mice injected with CTR cells developed large tumours, on the contrary, MLH1 knockout cells did not form tumours or developed very small lesions (FIG. 2B). PDAC cells injected in immune-deficient mice showed the same rate of engraftment confirming that the effects seen in immune-proficient mice were not related to impaired growth of clones (FIG. 2C). The percentage of CD8 T cells was clearly higher in MLH-1 KO clones confirming that neo-antigens post MLH-1 editing were involved in the recruitment of potential cytotoxic T cells (FIG. 2D).

The impact of the inactivation of mismatch repair on already established tumours was investigated. Since MLH1 knock-out cells do not grow, or grow poorly, in syngeneic immunocompetent mice they were first raised in immune-deficient mice until tumours reached 2000 mm³ in size, at which point lesions (2 mm per side) were transplanted in syngeneic BalbC recipients. Under these conditions, MLH1 knock-out colorectal cancer cells continued to grow in recipient animals (FIG. 3A). It was reasoned that this situation might recapitulate the clinical settings in which colorectal cancer patients typically receive immunotherapy, i.e. when tumours are fully established.¹⁷ Mice bearing MMR-proficient and MMR-deficient tumors were therefore treated with a combination of anti-PD1 and anti-CTLA4 antibodies. The results were striking, while control tumours continued to grow, their MMR-deficient counterparts regressed (FIG. 3A). In the majority of cases the therapy was curative as the lesions disappeared and the mice survived for more than three months without recurrences (FIG. 9B). At the end of the experiment a subset of the animals were sacrificed and histological analysis showed complete pathological response (CPR).

To gather insights into the molecular mechanisms responsible for these findings MHCI expression in the cell models was verified. Expression of MHCI was comparable among MMR proficient and deficient cells (FIG. 10). Next histological and FACS analyses on MMR-proficient and MMR-deficient tumours established in syngeneic mice that received isotype-specific control antibodies were conducted. Of note, MLH1 knock-out tumours displayed high levels of immune-infiltrate as compared with the control (FIG. 3B). Specifically, increased levels of CD8 T cells were found in MMR-deficient, but not in MMR-proficient, tumours that received isotype control antibodies. The experiment was repeated such that samples of MMR-deficient and MMR-proficient tumours were collected at an early time point after anti PD-1 and CTLA-4 combinatorial treatment. CD8 T cells were found to be preferentially increased in MLH1 knock-out clones as compared to controls (FIG. 3B). The same was obtained after immunofluorescence staining of CD8 cells in tumour samples (FIG. 3C). In order to test the hypothesis that T cells might be responsible for the tumour formation phenotype observed, the injection of MMR-deficient cells in the presence of anti CD8 antibodies was repeated, isotype matched antibodies serving as controls. The results were unambiguous, MMR deficient cells readily formed tumours in syngeneic mice only when CD8 T cells were concomitantly suppressed (FIG. 3D). Depletion of CD8 in MLH-1 proficient tumor bearing mice increased tumour growth (FIG. 9C).

Several reports indicate that tumours with high mutational burden (such as melanoma and lung cancers) preferentially respond to immunotherapy.^{13,18-21} Notably however a large fraction of hyper-mutated tumours have unfavorable prognosis and do not respond to immune-modulators.^{14,17}

Exome sequencing of parental cells and of matched normal (germline) DNA revealed that CT26 and MC38, display high mutational loads, 150 and 129 mutations/megabase of DNA respectively (FIG. 4A). RNA seq analysis indicated that a large proportion of mutated genes are transcribed and therefore can act as neo-antigens. These results are consistent with previous reports and likely reflect the origin of CT26 and MC38 cells which were obtained from mice treated with a carcinogen known to be mutagenic.²² When classified based on mutational load levels measured in human cancers, CT26 tumours would be considered hypermutated. To address the question of why CT26 tumours would grow in syngeneic mice that were never exposed to these cells if high levels of neo-antigens cause cancer-cell engagement by the immune system, the mutational loads of MMR deficient cells were measured. It was found that inactivation of MLH1 further increased the mutational burden of CT26 (from 150 to 247-352 mutations/Mb) and MC38 (from 129 to 190-250 mutations/Mb). While it is possible that this increase is sufficient to initiate the immune response other possibilities were considered. It was postulated that MSI tumours not only have high numbers of mutations due to ineffective DNA repair, but also that their mutational landscapes fluctuate continuously as a result. To test this formally exome sequencing from cells collected longitudinally at distinct time-points was performed. As shown in FIG. 4, in MMR cells the mutation (and therefore the corresponding neo-antigen) profiles dynamically evolve over time. It is therefore proposed that a critical feature, which renders MMR-deficient cancer likely to respond to immunotherapy, is that their genomic landscape rapidly and dynamically evolves. This leads to the continuous emergence of novel mutations, which are progressively and repeatedly engaged by the immune system. This possibility would explain why clinical incidences of MMR-deficient tumours generally have a more favourable prognosis and tend to remain under control by the immune system for longer periods of time.

The present inventors propose that evasion of immune surveillance in MMR tumours is counterbalanced by dynamic emergence of new antigens that are engaged by new pools of T cells. As previously discussed inactivation of tumour suppressor genes involved in DNA repair increase the mutation rate of cancer cells and this fuels cancer progression. Mutagenic agents are known to promote carcinogenesis. Therefore increasing the number of mutations in human cells is considered a tumour-promoting event.²³ It is reasoned that forced increase of the number of mutations in cancer cells could be (paradoxically) beneficial for therapeutic purposes. However it is postulated that the mutational increase would have to be dynamic and not static. To test this possibility cancer cells were treated with mutagenic agents that may or may not result in permanent inactivation of the DNA repair machinery. The present inventors and others previously reported that resistance to mutagenic agents can be associated with inactivation of MMR genes such as MLH1 and MSH2.²⁴ A pharmacological screen was designed to identify anticancer agents that preferentially affect MMR proficient cells as compared to their MSI counterpart. For the screen FDA approved anticancer drugs were selected that are known to alkylate DNA and/or impair DNA replication (Table 2). Functional assays showed that MMR deficient cells are either not affected or more resistant to the anticancer agents that were tested (FIG. 5A). However colorectal and breast MMR proficient cells displayed preferential sensitivity to temozolomide (TMZ) as compared to their MSI counterpart. Temozolomide is a well know chemotherapeutic agent which is used for treatment of several tumour types and triggers DNA damage.^{25,26} It has been previously shown that TMZ exposure affects DNA repair and treatment with TMZ can result in MMR inactivation.²⁷ CT26 and MC38 MMR proficient cells were treated with temozolomide until resistant populations emerged. Exome analysis revealed that exposure to TMZ increases mutational loads to levels comparable to those achieved by inactivation of MMR (FIG. 5B). CT26 and MC38 cells were then injected in the corresponding syngeneic mice. TMZ resistant CT26 cells readily formed tumours and grew at rates comparable to their parental counterparts (FIG. 5C). However MC38 resistant to temozolomide did not form tumours. The MMR status of TMZ resistant CT26 and MC38 cells was therefore assessed. Most notably, MC38 but not CT26 cells displayed MMR deficiency as measured by microsatellite instability assays. It was further found that MLH1 expression was dramatically reduced in MC38 (but not CT26) cells (FIG. 5D). These results indicate that exposure to alkylating agents increases the mutational load of cancer cells, however this per se is not sufficient to drive tumour rejection in vivo.

TABLE 3

| Drug | Mechanism of action |
|---|---|
| Oxaliplatin | Platinum-based agents |
| Cisplatin | Platinum-based agents |
| SN38 | inhibitors of Topoisomerase I |
| Bendomusitne | Alkylating agents |
| Lomustine | Alkylating agents |
| Carmustine | Alkylating agents |
| Temozolomide | Alkylating agents |
| Chlorambucil | Alkylating agents |
| Gemcitabine | Nucleoside analog |
| Pemetrexed | Antimetabolite |
| 5 FU | Antimetabolite |

When considered together these findings have several implications. First, extensive efforts have been placed at developing drugs capable of restoring the function of tumour suppressor proteins in the hope they could act as anticancer agents. However the present inventors' data indicate that permanent inactivation (rather than reactivation) of tumour suppressor genes or gene products could instead be pursued for therapeutic purposes. The rationale for this unconventional approach is based on the concept that dynamic rather than static increases of the number of mutations in cancer cells can result in cell-based immune responses.

Secondly, immune-modulators such as PD-1 and PDL-1 inhibitors are effective only in a subset of cancer patients. Based on the results presented herein it is possible that patients that benefit from immunotherapy for an extended period of time have DNA repair defects that result in a dynamic hypermutation state. In colorectal cancer these populations mainly overlap with individuals carrying defects in MMR and polymerase genes. These genes are not frequently altered in melanoma and lung cancer, however a subset of these patients does have prolonged response to immune-blockade (Topalian, S. L. et al. N Engl J Med 366, 2443-2454, (2012)). It is conceivable that some of the melanoma and lung cancer patients that have outstanding and long lasting benefit from immune-modulators also carry molecular alterations that lead to a dynamic hyper-mutation state.

Another implication of these results is that an increase in dynamic mutational loads, which leads to continuous renewal of neoantigens can be induced pharmacologically and this can lead to effective immunosurveillance. In this respect, the results we obtained with temozolomide (a commonly used chemotherapeutic agent) suggest that drugs leading to inactivation of DNA repair functions might be systemically tolerable. The focus of the data presented herein is on MLH1 that is involved in mismatch repair, however other (tumour suppressor) genes could also be targeted, with the goal of promoting dynamic mutational load. For example one could envision drug-induced inactivation of DNA polymerases such as POLE and POLD. Components of the mismatch repair system and DNA polymerases are endowed with catalytic activity (ATPase and exonuclease activity respectively), and should therefore be amenable to pharmacological inhibition.

In conclusion the data presented by the present inventors suggest that inactivation of DNA mismatch repair leads to a dynamic hyper-mutation status that triggers long-lasting immune surveillance. These results offer the rationale for developing innovative anti-cancer therapies based on inactivation of DNA repair enzymes.

### Methods

### Mouse Models

All animal procedures were approved by the Ethical Commission of the University of Turin and by the Italian Ministry of Health, and they were performed in accordance with institutional guidelines. (4D.L.N.116, G.U., suppl. 40, 18 Feb. 1992) and international law and policies (EEC Council Directive 86/609, OJ L 358, 1, Dec. 12, 1987; NIH Guide for the Care and Use of Laboratory Animals, US National Research Council, 1996). Four- to six-week old C57/BL/6J, BalbC and NOD/SCID mice were obtained from Charles River (Calco, Como, Italy). In mice experiments cells were inoculated subcutaneously (5×10⁵ cells/mouse). Tumour growth as well as the health of mice was monitored until sacrifice. Tumour size was measured every 5 days and calculated using the formula: V=((d/2)2×(D/2))/2 (d=minor tumour axis; D=major tumour axis) and reported as tumour mass volume (mm³, mean±SEM of individual tumour volume).

The mouse model of pancreatic ductal adenocarcinoma was obtained by injecting orthotopically in a cohort of FVB/n syngeneic mice KrasLSL_G12D, p53R172H/+, Ink4a/Arfflox/+ cells (1×10³ cells/mouse) isolated as previously described. When injected into the pancreas of immuno-competent FVB/n mice, these lines were able to form tumours that recapitulated many feature of the spontaneous tumour microenvironment with an average latency of 3-4 weeks. Total tumour burden was quantified by measuring with a calliper and estimating the volume of individually excised macroscopic tumours (>1 mm³) with the formula described before.

### Cell Models

The CT26 and MC38 colorectal cancer cell lines were kindly provided from the laboratory of Maria Rescigno, PhD (European Institute of Oncology). The TS/A breast cancer cell line is an aggressive cell line established from the first in vivo transplant of a moderately differentiated mammary adenocarcinoma that arose spontaneously in a BALB/c mouse. TS/A cells were kindly provided by Federica Cavallo (Molecular Biotechnology Center, University of Torino, Italy). The Lewis Lung Carcinoma cell lines were purchased from ATCC. mPDAC cells were isolated from tumour-bearing PDAC mice. The pancreatic cancer GEMM model was from FVB/n background. The combined p53 point mutant and INK4a/Arf floxed mice, KIAPp48Cre, had the following genetic make-up: p48cre, KrasLSL_G12D, p53R172H/+, Ink4a/Arfflox/+. Note that the wild-type allele of the corresponding tumour suppressor gene is lost en route to tumour formation. CT26 and MC38 cell lines were expanded in vitro in RPM11640 10% FBS, plus glutamine, penicillin and streptomycin. TS/A LLC and PDAC were cultured in DMEM 10% FBS plus glutamine, penicillin and streptomycin.

### CRISPR/Cas9 Mediated Knockout of MLH1

To knockout the Mlh1 gene a genome editing system was used (all-in one and two vectors with a separate lentiviral construct that inducible delivers hSpCas9, a gift of Jonkers lab). For specific RNA-guide identification, with minimum off-targets effects, the software tools provided by the Zhang lab Web site were used (www.genome-engineering.org). Annealed sgRNA oligonucleotides targeting the murine Mlh1 were cloned into Bsmbl (Thermoscientific) restricted lentiCRISPR-v2 plasmid (from Addgene #52961) vector and lentiGuide-Puro (from Addgene #52963) as described previously.²⁹ Ligated plasmids were transformed into competent Stbl3 cells (Invitrogen). For each construct, 3-5 individual colonies were picked and grown in LB ampicillin media overnight. Plasmid from each colony was then isolated using a DNA minipreparation kit (Qiagen) and sequenced using hU6-forward primer to validate the correct integration orientation.

### Lentivirus Production and Infection

Lentiviral particles were packaged by the co-transfection of HEK293T cells with the viral vector and packaging plasmids pVSVg (AddGene #8454), psPAX2 (AddGene #12260) (Sanjana et al., Nat Method 2014). Transfection was achieved using CaCl₂ after which the cells were incubated for 48 hours. Supernatant from each well was then harvested, passed through a 0.22 μm filter to remove cell debris, and frozen as 1 mL aliquots at −80° C. The cells were infected with lentivirus at approximately 60% confluence in the presence of 8 μg/mL polybrene (Millipore). To select those cells transduced we used puromycin (P9620 Sigma Aldrich) treatment and Gentamicin (Gibco Life Technologies) in the case of the inducible vector. The induction of CAS9 was subsequent to 40H-tamoxifen (Sigma Aldrich) treatment (1 μg/ml) in vitro.

### Off-Target Effects in CRISPR/Cas9

To examine whether the CRISPR/Cas9 expression leads to off-target cutting, each sgRNA's top 20 off-target sites and at least three exonic off-targets were analysed. The analysis of amplicon-based NGS data revealed exclusively wild-type sequences at these predicted off-target sites. Thus, it is concluded that undesired off-target effects are negligible in the experimental setting of CRISPR/Cas9 expression²⁹.

### Treatments

The anti-mouse PD-1 (clone RMP1-14) and anti-mouse CTLA-4 (clone 9H10) antibodies were purchased from BioXell (USA). Mice were treated i.p. with 250 μg/mouse of anti PD-1 and 200 μg/mouse of anti CTLA-4. Treatments were administrated at days 3, 6 and 9 after injection. Anti PD-1 was given continuously every three days. Isotype controls (Rat IgG2a for PD-1 and polyclonal Syrian Hamster IgG for CTLA-4) were injected according to the same schedule. Anti-mouse CD8a (clone YTS 169.4) and the isotype rat IgG2b were used for depleting cytotoxic T cells in immunocompetent mice. Anti-mouse CD8a antibodies (200 μg/mouse) were injected i.p. the same day as tumour inoculation. After 2 and 3 days post tumour injection mice were treated with 100 μg/mouse of the depleting antibodies. FACS analysis was performed in order to control for the level of CD8a T cells in the bloodstream of mice without tumours. The in vivo inducible MLH1 knock-out was obtained by treating mice i.p. with tamoxifen. 10 mg/ml of tamoxifen (T5648 from Sigma-Aldrich) was dissolved in 1:10 of ethanol and 9:10 of peanut oil. Every mouse was injected daily with 100 μl of the drug for 5 days.

### Western Blots

For biochemical analysis, all cells were grown in media supplemented with 10% FBS. Total cellular proteins were extracted by solubilizing the cells in boiling SDS buffer (50 mM Tris-HCl [pH 7.5], 150 mM NaCl, and 1% SDS). Samples were boiled for 10 minutes and sonicated for 30 seconds. Extracts were clarified by centrifugation and normalized with the BCA Protein Assay Reagent Kit (Thermo Scientific). Western blot detection was performed with an enhanced chemiluminescence system (GE Healthcare) and peroxidase-conjugated secondary antibodies (Amersham). The following primary antibodies were used for Western blotting: anti mMLH-1, 1:5000 (epr3894 from AbCam), anti Actin (1-19) 1:2000 (from Santa Cruz Biotechnology).

### Immunophenotypic Analysis

Blood cells were collected from the tail vein of anesthetised mice. Mouse tumours were cut into small pieces, disaggregated with collagenase (1.5 mg/ml) and DNAse (100 μg/ml), and filtered through strainers. Cells (10⁶) were stained with specific antibodies and Zombie Violet Fixable Viability Kit (Biolegend). Flow cytometry was performed by FACS Dako instrument and FlowJo software. Phenotype analysis was performed with the following antibodies. PerCp-Rat CD45 (30F11), Rat APC CD11b (M1/70), Rat PE/Cy7 CD3 (17A2), FITC Rat CD4 (RM4-5) and PE Rat CD8 (YTS156.7.7).

### Exome and Bioinformatic Analysis

Genomic DNA was extracted using ReliaPrep™ gDNA KIT (Promega). Capture and enrichment of genomic DNA samples were performed by IntegraGen using the Agilent SureSelect Mouse All Exon Kit. Libraries were sequenced using Illumina HiSeq 4000. The bioinformatics analysis was performed at FPO Instituto di Ricovero e Cura a Carattere Scientifico (IRCCS-FPO) on sequencing raw data provided by IntegraGen. Raw data, in Fastq format, were demultiplexed using CASAVA 1.8 software as paired-end 75-bp reads. On average a median depth of 70× was observed, with more than 97% of the targeted-region covered by at least one read. Before further analysis, pair-end reads were aligned to the mouse reference, assembly mm10, using BWA-mem algorithm (Li, H. & Durbin, R. Bioinformatics 26, 589-595 (2010)). Then PCR duplicates were removed from the alignment files using the “rmdup” samtools command³⁰. Somatic variations were called subtracting germline variations found in BalbC and C57bl6 using a custom NGS pipeline³¹. Only positions present with minimum depth of 5× and supported by at least 1% allelic frequency were taken into account. To calculate the significance of the allele frequency we performed a Fischer test for each variant. The mutational burden was calculated considering only coding variants normalising on the targeted region. Neo-antigens were calculated starting from the annotation file of variations. The amino acid changes reported were used to reconstruct the peptide sequences within the codon changes. Mutated peptide sequences were properly trimmed and then were fed to NetMHC 4.0 software in order to predict neo-antigens.³² For each variation, only the predicted neo-antigen with the best rank was taken into account for generating suitable peptide output.

### Statistical Analysis

All data from the Cell Titer Glo^{R} (Promega) are presented as means±s.d. of at least three independent experiments, each with three experimental replicates. Mice experiments were performed with at least 4 mice per group. P values were calculated by Student's t-test.

### Example 2—Cell Based Assay for Modulators

In order to read out mismatch repair (MMR) in mammalian cells, an assay based on the activity of the NanoLuciferase (NanoLuc®, Promega) reporter enzyme was developed (the “CA₍₂₀₎-NanoLuc assay”). 20 copies of the CA dinucleotide repeat (referred to as “CA₍₂₀₎”) were cloned upstream of the NanoLuc coding sequence in order to place NanoLuc activity under the control of the MMR pathway. This CA₍₂₀₎ tract renders the NanoLuc coding sequence out of frame, and therefore there is therefore no enzyme expression and no activity. The CA₍₂₀₎ tract is, however, a sequence which is subject to frequent DNA replication errors, and is therefore reliant on the MMR pathway to repair any post-replicative DNA mismatches. In a MMR-competent cell, any errors are efficiently repaired, the NanoLuc coding sequence remains out of frame and thus reporter activity is low. If, however, MMR is inhibited either by a small molecule or by genetic loss of any of the MMR machinery, post-replicative errors may remain unrepaired, frameshift mutations may occur, and therefore some cells in a population will now express a functional NanoLuc protein. This is depicted in the cartoon shown in FIG. 11.

MMR inhibition can therefore be reported as an increase in NanoLuc® activity when a plasmid containing the CA₍₂₀₎ NanoLuc construct is transfected into cells. Since NanoLuc® is a highly processive enzyme with a large and linear dynamic range, it was predicted that only a small number of MMR errors would be required to generate a positive signal, making for a sensitive assay with a large signal to noise ratio.

This assay format was tested using HEK293A, FT and T cells. In these cells, the MLH1 promoter is hypermethylated to various extents, and therefore MLH1 expression is low. As shown in FIG. 12, when these cells are co-transfected with the CA₍₂₀₎-NanoLuc construct and a plasmid expressing wild-type human MLH1, the NanoLuc signal is almost completely suppressed. This demonstrates that the CA₍₂₀₎-NanoLuc plasmid reports on MLH1 activity and that MLH1 activity can be measured using this assay system.

To extend these findings, an ATPase dead mutant of human MLH1 was generated by mutating Glycine 67 to Arginine (G67R). This is a human Lynch Syndrome mutation and has been reported as ATPase dead in biochemical assays previously. In HEK293FT cells, the same, clear inhibition of NanoLuciferase activity as before with WT MLH1 was observed, but three different MLH1 G67R plasmids failed to inhibit the reporter activity, as shown in FIG. 13.

An assay for compounds is performed as follows:

HEK293FT cells are co-transfected with WT MLH1 and CA₍₂₀₎-NanoLuc plasmids, cells are re-plated into 96-well plates and then treated with a dose range of potential inhibitors. Active compounds will report an increase in reporter signal, rather than an inhibition. This is a distinct advantage when dealing with immature hit compounds which may cause cellular toxicity: in assay formats which report through loss of signal, signal reduction can often be due to cell death, leading to compounds falsely being called as hits.

### REFERENCES

1 Vogelstein, B. et al. Cancer genome landscapes. Science 339, 1546-1558, doi:10.1126/science.1235122 (2013).
2 Shojaee, S. et al. PTEN opposes negative selection and enables oncogenic transformation of pre-B cells. Nat Med, doi:10.1038/nm.4062 (2016).
3 Orthwein, A. et al. A mechanism for the suppression of homologous recombination in G1 cells. Nature 528, 422-426, doi:10.1038/nature16142 (2015).
4 Rayner, E. et al. A panoply of errors: polymerase proofreading domain mutations in cancer. Nat Rev Cancer 16, 71-81, doi:10.1038/nrc.2015.12 (2016).
5 Bronner, C. E. et al. Mutation in the DNA mismatch repair gene homologue hMLH1 is associated with hereditary non-polyposis colon cancer. Nature 368, 258-261, doi:10.1038/368258a0 (1994).
6 Koornstra, J. J. et al. Management of extracolonic tumours in patients with Lynch syndrome. Lancet Oncol 10, 400-408, doi:10.1016/51470-2045(09)70041-5 (2009).
7 Lax, S. F., Kendall, B., Tashiro, H., Slebos, R. J. & Hedrick, L. The frequency of p53, K-ras mutations, and microsatellite instability differs in uterine endometrioid and serous carcinoma: evidence of distinct molecular genetic pathways. Cancer 88, 814-824 (2000).
8 Xiao, X., Melton, D. W. & Gourley, C. Mismatch repair deficiency in ovarian cancer—molecular characteristics and clinical implications. Gynecol Oncol 132, 506-512, doi:10.1016/j.ygyno.2013.12.003 (2014).
9 Okuda, T. et al. Genetics of endometrial cancers. Obstet Gynecol Int 2010, 984013, doi:10.1155/2010/984013 (2010).
10 Ponti, G., Castellsagué., Ruini, C., Percesepe, A. & Tomasi, A. Mismatch repair genes founder mutations and cancer susceptibility in Lynch syndrome. Clin Genet 87, 507-516, doi:10.1111/cge.12529 (2015).
11 Vilar, E. & Gruber, S. B. Microsatellite instability in colorectal cancer—the stable evidence. Nat Rev Clin Oncol 7, 153-162, doi:10.1038/nrclinonc.2009.237 (2010).
12 Rizvi, N. A. et al. Cancer immunology. Mutational landscape determines sensitivity to PD-1 blockade in non-small cell lung cancer. Science 348, 124-128, doi:10.1126/science.aaa1348 (2015).
13 Schumacher, T. N. & Schreiber, R. D. Neoantigens in cancer immunotherapy. Science 348, 69-74, doi:10.1126/science.aaa4971 (2015).
14 McGranahan, N. et al. Clonal neoantigens elicit T cell immunoreactivity and sensitivity to immune checkpoint blockade. Science, doi:10.1126/science.aaf1490 (2016).
15 Speroni, L. et al. Alternative site of implantation affects tumor malignancy and metastatic potential in mice: its comparison to the flank model. Cancer Biol Ther 8, 375-379 (2009).
16 Bardeesy, N. et al. Both p16(Ink4a) and the p19(Arf)-p53 pathway constrain progression of pancreatic adenocarcinoma in the mouse. Proc Natl Acad Sci USA 103, 5947-5952, doi:10.1073/pnas.0601273103 (2006).
17 Le, D. T. et al. PD-1 Blockade in Tumors with Mismatch-Repair Deficiency. N Engl J Med 372, 2509-2520, doi:10.1056/NEJMoa1500596 (2015).
18 Xiao, Y. & Freeman, G. J. The microsatellite instable subset of colorectal cancer is a particularly good candidate for checkpoint blockade immunotherapy. Cancer Discov 5, 16-18, doi:10.1158/2159-8290. CD-14-1397 (2015).
19 Llosa, N. J. et al. The vigorous immune microenvironment of microsatellite instable colon cancer is balanced by multiple counter-inhibitory checkpoints. Cancer Discov 5, 43-51, doi:10.1158/2159-8290. CD-14-0863 (2015).
20 Topalian, S. L. et al. Safety, activity, and immune correlates of anti-PD-1 antibody in cancer. N Engl J Med 366, 2443-2454, doi:10.1056/NEJMoa1200690 (2012).
21 Brahmer, J. R. et al. Safety and activity of anti-PD-L1 antibody in patients with advanced cancer. N Engl J Med 366, 2455-2465, doi:10.1056/NEJMoa1200694 (2012).
22 Lerner, W. A., Pearlstein, E., Ambrogio, C. & Karpatkin, S. A new mechanism for tumor induced platelet aggregation. Comparison with mechanisms shared by other tumor with possible pharmacologic strategy toward prevention of metastases. Int J Cancer 31, 463-469 (1983).
23 Castro-Giner, F., Ratcliffe, P. & Tomlinson, I. The mini-driver model of polygenic cancer evolution. Nat Rev Cancer 15, 680-685, doi:10.1038/nrc3999 (2015).
24 Bardelli, A. et al. Carcinogen-specific induction of genetic instability. Proc Natl Acad Sci USA 98, 5770-5775, doi:10.1073/pnas.081082898 (2001).
25 Chiappinelli, K. B., Zahnow, C. A., Ahuja, N. & Baylin, S. B. Combining Epigenetic and Immunotherapy to Combat Cancer. Cancer Res, doi:10.1158/0008-5472.CAN-15-2125 (2016).
26 Fink, D., Aebi, S. & Howell, S. B. The role of DNA mismatch repair in drug resistance. Clin Cancer Res 4, 1-6 (1998).
27 McFaline-Figueroa, J. L. et al. Minor Changes in Expression of the Mismatch Repair Protein MSH2 Exert a Major Impact on Glioblastoma Response to Temozolomide. Cancer Res 75, 3127-3138, doi:10.1158/0008-5472.CAN-14-3616 (2015).
28 Kilpivaara, O. & Aaltonen, L. A. Diagnostic cancer genome sequencing and the contribution of germline variants. Science 339, 1559-1562, doi:10.1126/science.1233899 (2013).
29 Sanjana, N. E., Shalem, O. & Zhang, F. Improved vectors and genome-wide libraries for CRISPR screening. Nat Methods 11, 783-784, doi:10.1038/nmeth.3047 (2014).
30 Li, H. et al. The Sequence Alignment/Map format and SAMtools. Bioinformatics 25, 2078-2079, doi:10.1093/bioinformatics/btp352 (2009).
31 Siravegna, G. et al. Clonal evolution and resistance to EGFR blockade in the blood of colorectal cancer patients. Nat Med, doi:10.1038/nm.3870 (2015).
32 Andreatta, M. & Nielsen, M. Gapped sequence alignment using artificial neural networks: application to the MHC class|system. Bioinformatics 32, 511-517, doi:10.1093/bioinformatics/btv639 (2016).

1. A method for treating cancer comprising:
a) providing i) a subject having cancerous cells, and ii) a modifier of a DNA repair or nucleic acid editing gene, or its protein product; and
b) treating said subject with said modifier wherein said treating reduces the number of cancerous cells in said subject.

2. A method as claimed in claim 1 wherein the modifier of a DNA repair or nucleic acid editing gene or its protein product is an activator.

3. A method as claimed in claim 2 wherein the DNA repair gene encodes a protein involved in translesion synthesis.

4. A method as claimed in claim 3 wherein the DNA repair gene is DNA-pol η, ι or κ.

5. A method as claimed in claim 2 wherein the nucleic acid editing gene encodes a protein involved in RNA or DNA editing.

6. A method as claimed in claim 5 wherein the nucleic acid editing enzyme is ADAR1, ADAR2, ADAR3, ADARB2-AS1, AICDA, APOBEC3D, APOBEC3B, APOBEC3C, APOBEC3G, APOBEC3F, APOBEC3A, APOBEC3H, APOBEC1, A1CF, APOBEC2, APOBEC4, APOBEC3AP1 or APOBEC3B-AS1.

7. A method as claimed in claim 1 wherein the modifier of a DNA repair or nucleic acid editing gene is an inactivator.

8. A method as claimed in claim 7 wherein the DNA repair gene is an MMR gene.

9. A method as claimed in claim 8 wherein the MMR gene is a MutL homologue.

10. A method as claimed in claim 9 wherein the MutL homologue is MLH1, MutLα, MutLβ, MutLγ, PMS1, PMS2 or MLH3.

11. A method as claimed in claim 7 wherein the nucleic acid editing enzyme is ADAR1, ADAR2, ADAR3, ADARB2-AS1, AICDA, APOBEC3D, APOBEC3B, APOBEC3C, APOBEC3G, APOBEC3F, APOBEC3A, APOBEC3H, APOBEC1, A1CF, APOBEC2, APOBEC4, APOBEC3AP1 or APOBEC3B-AS1.

12. A method as claimed in claim 1, wherein the modifier is a polypeptide, polynucleotide, antibody, peptide or small molecule compound.

13. A method as claimed in claim 1, wherein an inactivator is a molecule which provides inactivation through genome editing.

14. A method as claimed in claim 1, wherein the cancer is an MMR +ve cancer.

15. A method for treating cancer as claimed in claim 1, wherein the modifier of a DNA repair enzyme is provided in combination with a different cancer treatment.

16. A method as claimed in claim 15 wherein the different cancer treatment is one which inactivates MMR genes.

17. A method as claimed in claim 16 wherein the different cancer treatment is treatment with temozolomide or MNU or their derivatives.

18. A method as claimed in claim 15 wherein the different cancer treatment is an immunotherapy.

19. A method as claimed in claim 18 wherein the immunotherapy is an immune checkpoint inhibitor or combination of immune checkpoint inhibitors.

20. A method as claimed in claim 19 wherein the immune checkpoint inhibitor is an anti-PD1 antibody, an anti-CTLA-4 antibody, an anti-PDL-1 antibody or combinations thereof.

21. A method of treatment as claimed in claim 1 wherein the inactivator/inhibitor of a mismatch repair gene is temozolomide, MNU or their derivatives.

22. A method for screening for anti-cancer compounds comprising
a) providing cells expressing a DNA repair or nucleic acid editing gene;
b) incubating said cells in the presence of a test compound; and
c) measuring the rate of DNA or RNA mutation in the presence of a test compound;
d) wherein an increased rate of DNA or RNA mutation in cells in the presence of a test compound compared to that measured in cells in the absence of a test compound indicates a test compound is an anti-cancer compound.

23. A method as claimed in claim 22 wherein cells expressing a DNA repair gene are a human tumour cell line.

24. A method as claimed in claim 22 wherein the DNA repair gene is MLH1, MutLα, MutLβ, MutLγ, PMS1, PMS2 or MLH3.

25. A method as claimed in claim 22 or claim 23 wherein the nucleic acid editing enzyme is ADAR1, ADAR2, ADAR3, ADARB2-AS1, AICDA, APOBEC3D, APOBEC3B, APOBEC3C, APOBEC3G, APOBEC3F, APOBEC3A, APOBEC3H, APOBEC1, A1CF, APOBEC2, APOBEC4, APOBEC3AP1 or APOBEC3B-AS1.

26. A method of identifying a patient having a tumour suitable for treatment by immunotherapy comprising:
a) taking a sample of said tumour,
b) analysing said sample to determine the sequence of a DNA repair or nucleic acid editing gene, and
c) comparing the sequence of said DNA repair or nucleic acid editing gene in a tumour sample with the sequence in a non-tumour sample,
wherein a defect in the sequence of said DNA repair or nucleic acid editing gene in the tumour sample compared to the sequence of said gene in a non-tumour sample is indicative that said patient has a tumour suitable for treatment by immunotherapy.

27. A method for screening for a modifier of a DNA repair or nucleic acid editing gene, or its protein product, comprising:
a) providing a construct wherein comprising a simple nucleotide sequence cloned upstream of a reporter coding sequence such that the reporter coding sequence is out of frame;
b) transfecting cells with the construct of a) in combination with a construct comprising a DNA repair or nucleic acid editing gene;
c) incubating said cells in the presence of a test compound; and
d) measuring a signal from the reporter construct;
e) wherein an increase in signal from the reporter construct in the presence of the test compound compared to the signal in the absence of the test compound indicates that the test compound is a modifier of said DNA repair or nucleic acid editing gene, or its protein product.

28. A method as claimed in claim 27 wherein the simple nucleotide sequence is a CA dinucleotide repeat, preferably CA₍₂₀₎.

29. A method as claimed in claim 27 wherein the DNA repair or nucleic acid editing gene in step b) is MLH1.

30. An inactivator of MLH-1 comprising a CRISPR construct as described herein.
